(19) United States
(12) Patent Application Publication
(10) Pub. No.: US 2023/0173089 A1
(43) Pub. Date: Jun. 8, 2023

(54) Q-ER PEPTIDE

(71) Applicant: Biotempt B.V., The Hague (NL)

(72) Inventor: Gert Wensvoort, Koekange (NL)

(21) Appl. No.: 17/639,018

(22) PCT Filed: Aug. 28, 2020
(86) PCT No.: PCT/NL2020/050535

§ 371 Date: Feb. 28, 2022

(30) Foreign Application Priority Data
Aug. 30, 2019 (EP) 19194645.8

(51) Int. Cl. A61K 47/68; A61K 9/127; A61K 38/28; A61P 3/00

(52) U.S. Cl. CPC A61K 47/6849; A61K 9/127; A61K 38/28; A61K 47/6811; A61P 3/00

(57) ABSTRACT

Means and methods for treating diseases involving autophagy by cells, which is involved in mechanisms of tissue repair, vascular permeability and immune responses. Described are methods and means to target the elastin receptor complex specifically and to provide molecules and compositions comprising a specific targeting agent as well as amino acid compositions that are involved in the pathway of autophagy and the diseases related thereto. Also, peptide-drug development, in particular, to (the improvement of) autophagy inhibiting amino acid containing peptides, more, in particular, glutamine-containing peptides and/or glutamine and other autophagy modulating amino acid containing compositions useful in the treatment of vascular and inflammatory conditions. Improvement of glutamine peptides useful in the treatment of diabetic, vascular and/or inflammatory conditions. A Q-ER peptide, comprising a synthetic peptide or functional analogue thereof provided with at least one PG-domain amino acid motif xGxxPG or functional equivalent thereof, the PG-domain motif allowing targeting of the peptide to the elastin receptor complex (ER), wherein at least one amino acid at position x is selected from A, Q, G, V, L, I, P, and R.

### CROSS-REFERENCE TO RELATED APPLICATIONS

This application is a national phase entry under 35 U.S.C. § 371 of International Patent Application PCT/NL2020/050535, filed Aug. 28, 2020, designating the United States of America and published as International Patent Publication WO 2021/040526 A1 on Mar. 4, 2021, which claims the benefit under Article 8 of the Patent Cooperation Treaty to European Patent Application Serial No. 19194645.8, filed Aug. 30, 2019.

### TECHNICAL FIELD

This disclosure relates to means and methods for treating diseases that involve autophagy by cells, which process according to the disclosure is involved in mechanisms of tissue repair, vascular permeability and immune responses. The disclosure provides methods and means to target the elastin receptor complex specifically and to provide molecules and compositions comprising a specific targeting agent as well as amino acid compositions that are involved in the pathway of autophagy and the diseases related thereto. The disclosure also relates to peptide-drug development, in particular, to (the improvement of) autophagy inhibiting amino acid containing peptides, more, in particular, glutamine-containing peptides and/or glutamine and other autophagy modulating amino acid containing compositions useful in the treatment of vascular and inflammatory conditions.

### BACKGROUND

Glutamine (Gln, Q) is the most abundant free amino acid in the plasma and tissue pool. It serves as an important fuel source for rapidly dividing cells, especially leucocytes and enterocytes. Glutamine is the most abundant nonessential amino acid in the body and in states of stress it becomes a conditionally essential amino acid. It is the preferred fuel source for the small bowel enterocyte, which is thought to help maintain its structure and function during times of stress. In septic and malnourished patients, muscle glutamine is depleted, and it is hypothesized that in these patients the availability of glutamine in lymphocytes and the gut is reduced, resulting in increased risk of sepsis. Although enteral formulas designed to improve immunity have given mixed results, glutamine supplementation has been shown not to be harmful and in fact reduced complications in patients with bone marrow transplantation, after surgery, and in patients with critical illness and severe burns.

Studies using parenteral glutamine have generally been more positive than those employing enteral glutamine. Although it is considered a non-essential amino acid, many studies showed that Gln has beneficial tissue-regenerating properties and is considered conditionally essential for patients with catabolic conditions [J Nutr 131: 2543S-2549S discussion 2550S-2541S.; Nutr Rev 48: 297-309. doi: 10.1111/j.1753-4887.1990.tb02967.x; Yonsei Med J 52: 892-897. doi: 10.3349/ymj.2011.52.6.892; Lancet 336: 523-525. doi: 10.1016/0140-6736(90)92083-t; PLoSONE 9(1): e84410.doi:10.1371/journal.pone.0084410]. Shiomi et al. [Inflamm Bowel Dis 17: 2261-2274. doi: 10.1002/ibd.21616] reported that Gln levels of serum and colon tissues were significantly lower in the acute phase of colonic inflammation, and Gln supplementation attenuated the degree of microscopic injury induced by dextran sulfate sodium (DSS). Also, glutamine and alanyl-glutamine dipeptide reduce vascular permeability with mesenteric plasma extravasation, leukocyte adhesion and tumor necrosis factor-α (TNF-α) release during experimental endotoxemia [Scheibe, Ricardo et al., 2009, - 60 Suppl 8 Journal of physiology and pharmacology: an official journal of the Polish Physiological Society].

Glutamine-containing di-peptides, such as alanyl-glutamine (in one-letter-code AQ, tradename DIPEPTIVIN® ), glycyl-glutamine (GQ), leucyl-glutamine (LQ), valyl-glutamine (VQ), isoleucyl-glutamine (IQ), and cysteinyl-glutamine (CQ), have earlier been found useful in the treatment of various conditions (see also US20050059610). Tri- and tetrapeptide formulations comprising glutamine (such as LQG, SEQ ID NO:1 (LQGV), AQG, or SEQ ID NO:2 (AQGV), see also WO2012112048) are, above the di-peptides listed, advantageously used in methods and pharmaceutical compositions to treat severe systemic conditions. The tri-and tetra-peptides are synthetic linear glutamine-containing peptides derived from the beta-human chorionic gonadotropin hormone, which have tissue-protective effects in animal studies.

For example, the tetrapeptide with SEQ ID NO:1 (LQGV) has been shown (van den Berg et al., Crit Care Med 39: 126-134.) to reduce mortality in a murine polymicrobial sepsis model. LQGV (at 5 mg/kg bodyweight) significantly improved survival from 20% to 50% during the first 5 days after moderate cecal ligation and puncture. This was associated with reduced cytokine and E-selectin levels in peritoneal lavage fluid, lungs, and, to a lesser extent, in plasma. SEQ ID NO: 1 (LQGV) treatment also reduced pulmonary nuclear factor-κB activation and pulmonary damage. In a severe cecal ligation and puncture model, the tetrapeptide with SEQ ID NO: 1 (LQGV) (at 5 mg/kg bodyweight) combined with fluid resuscitation and antibiotics resulted in significantly better survival (70%) than that observed with fluid resuscitation and antibiotics alone (30%).

Also, the tetrapeptide with SEQ ID NO:2 (AQGV) has been shown (Gueller et al, PLoS One. 2015 Jan 24;10(1): e0115709. doi: 10.1371/journal.pone.0115709. eCollection 2015) to improve survival and attenuate loss of kidney function in mouse models renal ischemia/ reperfusion injury (IRI) and of ischemia-induced delayed graft function after allogenic kidney transplantation. IRI was induced in male C57B1/6N mice by transient bilateral renal pedicle clamping for 35 min. Treatment with SEQ ID NO:2 (AQGV) (20-50 mg/kg twice daily i.p. for four consecutive days) was initiated 24 hours after IRI when acute kidney injury (AKI) was already established. The treatment resulted in markedly improved survival in a dose dependent manner. Acute tubular injury two days after IRI was diminished and tubular epithelial cell proliferation was significantly enhanced by SEQ ID NO:2 (AQGV) treatment. Furthermore, CTGF up-regulation, a marker of post-ischemic fibrosis, at four weeks after IRI was significantly less in SEQ ID NO:2 (AQGV) treated renal tissue. Next, SEQ ID NO:2 (AQGV) treatment was tested in a model of ischemia-induced delayed graft function after allogenic kidney transplantation. The recipients were treated with SEQ ID NO:2 (AQGV) (50 mg/kg) twice daily i.p., which improved renal function and allograft survival by attenuating ischemic allograft damage.

The tetrapeptide with SEQ ID NO:2 (AQGV) has also been shown (Groenendael et al., Intensive Care Medicine Experimental 2016, 4(Suppl 1):A132) to be safe and have significant beneficial immunomodulatory effects in an experimental model of systemic inflammatory response syndrome (SIRS) in humans. SIRS can lead to pronounced tissue damage and is a frequent cause of multi-organ failure and mortality in intensive care units. SIRS can be elicited by a variety of insults, such as sepsis, trauma and major surgery, and no specific therapy is currently in routine use. To investigate the tolerability, safety and immunomodulatory effects of the tetrapeptide with SEQ ID NO:2 (AQGV) in humans, a double blind, placebo controlled, dose-escalating randomized clinical trial in 60 healthy volunteers has been conducted. The study was carried out in two phases. In the first phase (n = 24), safety and tolerability was established for escalating doses of the peptide (30, 90, and 180 mg/kg). In the second phase (n = 36), the same doses were used to assess the effects of the peptide on systemic inflammation (SIRS) during experimental human endotoxemia. At t = 0 hours, 2 ng/kg E. coli endotoxin was administered i.v. followed by a 2-hour continuous i.v. infusion of tetrapeptide with SEQ ID NO:2 (AQGV) or placebo. Levels of circulating cytokines and adhesion molecules as well as body temperature and flu-like symptoms were assessed.

The tetrapeptide with SEQ ID NO:2 (AQGV) was well tolerated and showed an excellent safety profile. Treatment with at 180 mg/kg (the highest dose) of the peptide, but not with the lower doses tested, resulted in a significant attenuation of the endotoxin induced increase in plasma levels of IL-6, IL-8, IL-1RA, MCP-1, MIP-1α, and MIP-1β and the adhesion molecule VCAM-1. Furthermore, the highest dose reduced fever and flu-like symptoms. It was concluded that administration of the tetrapeptide with SEQ ID NO:2 (AQGV) is safe and results in attenuation of the systemic inflammatory response in humans. However, a drawback of the here above discussed di-, tri- and tetra-peptides for parenteral application is that they run the chance of being rapidly hydrolyzed in the blood before they have reached their target cells and can exert their beneficial actions. This rapid loss of peptide necessitates using high doses and long applications times to obtain the desired beneficial effects.

### BRIEF SUMMARY

According to the instant disclosure, the amino acids that have the beneficial effects are targeted to the cells in which they can have their beneficial effects, in particular, by targeting the elastin receptor complex through any specific means. Preferably the targeting means enables internalization of the amino acids and when the amino acids are provided in an oligopeptide format the internalization typically results in the oligopeptide being delivered to a lysosome (generally, and herein, also called autophagosome). Thus the disclosure provides a method for lowering autophagy, comprising targeting cells having an elastin receptor complex associated with their surface with a molecule specifically recognizing the complex, whereby the molecule is provided with a source of autophagy inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). The targeting then results in delivering the source, as a package of autophagy inhibiting amino acids, to the cell, where the molecule provided with the source or package is, for example, taken up by common endocytosis and/or phagocytosis, and then hydrolyzed into its collection of constituent, preferably autophagy inhibiting, amino acids in lysosomes (autophagosomes), and individual amino acids are released in the cytosol of the cell. In this way the mechanistic target of rapamycine (mTOR) is activated by the collection of autophagy inhibiting amino acid in the package selected for targeting of the Q-ER peptide to the cell. It is preferred that the molecule comprises a peptide (herein also termed Q-ER peptide) comprising at least 7 amino acids and at most 30 amino acids comprising a sequence of the formula ϕn xGxxPG, or xGxxPG ϕn, or ϕn xGxxPG ϕm wherein x is a naturally occurring amino acid, ϕ is an autophagy inhibiting amino acid and n = an integer from 1 to 24 and m is an integer from 1-23, whereby n+m is no greater than 24. In a preferred embodiment, n+m is no greater than 16, more preferably no greater than 12, more preferably no greater than 8. In a preferred embodiment, the disclosure provides a method wherein the molecule comprises a Q-ER peptide according to the disclosure wherein ϕn and/or ϕm comprise a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group SEQ ID NO: 1 (LQGV) and SEQ ID NO:2 (AQGV), preferably selected from the group AQ, LQ, GQ, AQL, LQL, GQL, PLQ, LQG, and AQG. It is preferred that the molecule is connected to the peptide through a peptide bond.

Also provided is a peptide comprising at least 7 amino acids and at most 30 amino acids, and wherein the peptide comprises a sequence of the formula ϕn xGxxPG, or xGxxPG ϕn, or ϕn xGxxPG ϕm wherein x is a naturally occurring amino acid, ϕ is an autophagy inhibiting amino acid and n = an integer from 1 to 24 and m is an integer from 1-23, whereby n+m is no greater than 24.

The disclosure also provides a method for producing a Q-ER peptide according to the disclosure comprising synthesizing the peptide with an automated peptide synthesizer, and provides a Q-ER peptide obtainable by synthesizing with an automated peptide synthesizer and use of the Q-ER peptide for lowering autophagy of cells of a subject, in particular, when the subject is deemed to be in need of such treatment. There is a certain balance to be achieved in the size of the oligopeptides provided by the disclosure. For speed of uptake and lower risk of immune responses smaller sizes are preferred, for half-life reasons and amount of autophagy lowering amino acids delivered longer sequences are preferred. Depending on the condition to be treated and the doses considered acceptable the skilled person will be capable of determining the right size of the oligopeptide or combinations of different sizes, optionally with additional autophagy lowering amino acids provided concomitantly (e.g., through conjugation to vehicles comprising the additional amino acids).

Therewith, the disclosure provides a method to regulate central cellular events that involve the mechanistic target of rapamycin (mTOR) pathway (Liu and Sabatini, Nature Reviews Molecular Cell Biology volume 21, pages 183-203(2020)) The mTOR pathway integrates a diverse set of environmental cues, such as growth factor signals and nutritional status, to direct eukaryotic cell growth. Over the past two and a half decades, mapping of the mTOR signaling landscape has revealed that mTOR controls biomass accumulation and metabolism by modulating key cellular processes, including protein synthesis and autophagy, balancing mTOR activated proteogenesis versus proteolytic autophagy in a cell, respectively. The disclosure provides delivering a source of autophagy inhibiting amino acids to a targeted cell, after which the cell, and, in particular, the lysosomal compartment of the cell, is provided with the source of autophagy inhibition amino acids through endocytosis or phagocytosis and amino acids are liberated (e.g., through enzymatic hydrolysis) in the compartment and become available for cytosolic routing.

Given mTOR’s pathway central role in maintaining cellular and physiological homeostasis, dysregulation of mTOR signaling has been implicated in many disorders with a general cellular origin, such as metabolic disorders as diabetes, neurodegeneration, cancer, inflammation and ageing. In particular, the mechanistic target of rapamycin complex I (mTORC1) is a central regulator of cellular and organismal growth, and this pathway is implicated in the pathogenesis of many animal- and human diseases. mTORC1 promotes growth in response to the availability of nutrients, such as amino acids in lysosome and transferred to cytosol, which drive mTORC1 to the lysosomal surface, its site of activation. According to the disclosure some amino acids activate mTOR more than others, and therewith inhibit autophagy or stimulate proteogenesis more than others, in particular, amino acids selected from the group of alanine (in one letter code: A), glutamine (Q), glycine (G), valine (V), leucine (L), isoleucine (I), proline (P) and arginine (R) are known to inhibit autophagy more than other natural occurring amino acids. The disclosure now provides targeting collections or strings of such selected autophagy inhibiting amino acids delivered at cells that help an organism tackle or combat disease by curing tissue defects central to the health of an organism, in particular, of a human organism; the cells in and around the vascular system that are central in curative activity and relate to vascular integrity or permeability, to tissue repair and to innate and adaptive immune responses, all cells that, in various ways, are involved in curing an organism from damage resulting from insult, injury, infection, metabolic alteration and cellular degeneration.

Central to the delivery of such sources of autophagy inhibiting amino acids to cells and tissues in curative need, the disclosure provides use of targeted delivery of a collection or source of such amino acids to cells having an elastin receptor complex associated with their surface, as these cells (examples of cells having or carrying a surface-associated elastin receptor complex in at least a part of their life cycle are red and white blood cells, vascular endothelial cells, smooth muscle cells and fibroblasts) are typically involved in curative activities that benefit from lowered and at least partly inhibited autophagy and likewise increased and improved mTOR mediated proteogenesis.

Also provided is a method for modifying vascular permeability, comprising targeting cells having an elastin receptor complex associated with their surface with a molecule specifically recognizing the complex, whereby the molecule is provided with a source of autophagy inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). Increased vascular permeability is, for example, governing fluid and white blood cell (leukocyte) extravasation, that is initially required in acute inflammations, but that in a later stage typically needs inhibition or reduction (i.e., lower permeability, or return to original vascular integrity) to allow for repair of tissue after, for example, inflammation has had its function and tissue is set to regain its integrity and be healed. Therewith, the disclosure is also providing a method for improving or promoting tissue repair, comprising targeting cells having an elastin receptor complex associated with their surface with a molecule specifically recognizing the complex, whereby the molecule is provided with a source of autophagy inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). Restoring tissue integrity, in particular, is beneficial when acute immune responses need to be dampened and to switch the immune response toward a curative and tissue repairing response. The disclosure therewith provides a method for modulating an immune response, comprising targeting cells having an elastin receptor complex associated with their surface with a molecule specifically recognizing the complex, whereby the molecule is provided with a source of autophagy inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P).

It is preferred herein that the molecule recognizing the complex has an elastin peptide motif represented by xGxxPG, wherein x represents a naturally occurring amino acid, however, the elastin receptor complex has a rather promiscuous nature, often also recognizing a functionally related motif xGxPG or xGxxPx, in particular, when the amino acid following P can adapt to a VIII beta-turn that facilitates recognition by the elastin receptor complex. It is preferred that the source of autophagy inhibiting amino acids is a peptide comprising the autophagy inhibiting amino acids. For such purpose, it is preferred that the peptide comprising the autophagy inhibiting amino acids comprises a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group SEQ ID NO: 1 (LQGV) and SEQ ID NO:2 (AQGV), preferably selected from the group AQ, LQ, GQ, AQL, LQL, GQL, PLQ, LQG, AQG. It is moreover preferred that the elastin receptor complex binding motif xGxxPG is connected to the peptide comprising the autophagy inhibiting amino acids by a peptide bond. These molecules of the disclosure can be simply produced by peptide synthesizers and can be readily degraded once in the lysosome to produce the autophagy inhibiting amino acids.

The disclosure also provides a molecule specifically recognizing an elastin receptor complex for use in lowering autophagy, the molecule comprising a source of autophagy inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). Preferably, such as molecule as provided herein comprises a peptide (herein also termed Q-ER peptide) comprising at least 7 amino acids and at most 30 amino acids comprising a sequence of the formula ϕn xGxxPG, or xGxxPG ϕn, or ϕn xGxxPG ϕm wherein x is a naturally occurring amino acid, ϕ is an autophagy inhibiting amino acid and n = an integer from 1 to 24 and m is an integer from 1-23, whereby n+m is no greater than 24. In a preferred embodiment, n+m is no greater than 16, more preferably no greater than 12, more preferably no greater than 8. In a preferred embodiment, the disclosure provides a Q-ER peptide according to the disclosure wherein ϕn and/or ϕm comprise a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group SEQ ID NO: 1 (LQGV) and SEQ ID NO:2 (AQGV), preferably selected from the group AQ, LQ, GQ, AQL, LQL, GQL, PLQ, LQG, AQG

The disclosure also provides a molecule specifically recognizing an elastin receptor complex for use in the modulation of an immune response, the molecule comprising a source of autophagy inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). Preferably, such as molecule as provided herein comprises a peptide (herein also termed Q-ER peptide) comprising at least 7 amino acids and at most 30 amino acids comprising a sequence of the formula ϕn xGxxPG, or xGxxPG ϕn, or ϕn xGxxPG ϕm wherein x is a naturally occurring amino acid, ϕ is an autophagy inhibiting amino acid and n = an integer from 1 to 24 and m is an integer from 1-23, whereby n+m is no greater than 24. In a preferred embodiment, n+m is no greater than 16, more preferably no greater than 12, more preferably no greater than 8. In a preferred embodiment, the disclosure provides a Q-ER peptide according to the disclosure wherein ϕn and/or ϕm comprise a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group SEQ ID NO: 1 (LQGV) and SEQ ID NO:2 (AQGV), preferably selected from the group AQ, LQ, GQ, AQL, LQL, GQL, PLQ, LQG, AQG

The disclosure also provides a molecule specifically recognizing an elastin receptor complex for use in improving tissue repair, the molecule comprising a source of autophagy inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). Preferably, such as molecule as provided herein comprises a peptide (herein also termed Q-ER peptide) comprising at least 7 amino acids and at most 30 amino acids comprising a sequence of the formula ϕn xGxxPG, or xGxxPG ϕn, or ϕn xGxxPG ϕm wherein x is a naturally occurring amino acid, ϕ is an autophagy inhibiting amino acid and n = an integer from 1 to 24 and m is an integer from 1-23, whereby n+m is no greater than 24. In a preferred embodiment, n+m is no greater than 16, more preferably no greater than 12, more preferably no greater than 8. In a preferred embodiment, the disclosure provides a Q-ER peptide according to the disclosure wherein ϕn and/or ϕm comprise a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group SEQ ID NO: 1 (LQGV) and SEQ ID NO:2 (AQGV), preferably selected from the group AQ, LQ, GQ, AQL, LQL, GQL, PLQ, LQG, AQG

The disclosure also provides a molecule specifically recognizing an elastin receptor complex for use in modifying vascular permeability, the molecule comprising a source of autophagy inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). Preferably, such as molecule as provided herein comprises a peptide (herein also termed Q-ER peptide) comprising at least 7 amino acids and at most 30 amino acids comprising a sequence of the formula ϕn xGxxPG, or xGxxPG ϕn, or ϕn xGxxPG ϕm wherein x is a naturally occurring amino acid, ϕ is an autophagy inhibiting amino acid and n = an integer from 1 to 24 and m is an integer from 1-23, whereby n+m is no greater than 24. In a preferred embodiment, n+m is no greater than 16, more preferably no greater than 12, more preferably no greater than 8. In a preferred embodiment, the disclosure provides a Q-ER peptide according to the disclosure wherein ϕn and/or ϕm comprise a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group SEQ ID NO: 1 (LQGV) and SEQ ID NO:2 (AQGV), preferably selected from the group AQ, LQ, GQ, AQL, LQL, GQL, PLQ, LQG, AQG

Moreover, the disclosure also provides alternative modes of targeting cells having an elastin receptor complex associated with their surface with a molecule specifically recognizing the complex, whereby the molecule is provided with a source of autophagy inhibiting amino acids, selected from the group of alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). In one further embodiment, the disclosure provides a method for lowering autophagy, comprising targeting cells having an elastin receptor complex associated with their surface with a molecule specifically recognizing the complex, wherein the molecule recognizing the complex is an antibody-like molecule, preferably selected from IgG, IgM, single chain antibodies, FAB- or FAB′2-fragments. In principle any antibody-like molecule that can specifically recognize an elastin receptor complex may be used, whereby single chain formats (one polypeptide chain only) including at least one Vh or Vhh are preferred. These formats are preferred because they can be used as oligopeptide with the autophagy lowering amino acids bound to them through peptide bonds. In fact, where a Q-ER peptide is mentioned, typically Q-Vh and/or Q-Vhh should also be considered disclosed. Antibody-like molecules are general rapidly phagocytosed upon binding to their target and delivered in the lysosomal compartment, where the amino acid of that source can be further utilized for mTOR activation. Thus internalizing antibody-like molecules are preferred. It is preferred that the source of autophagy inhibiting amino acids is a peptide comprising a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group SEQ ID NO: 1 (LQGV) and SEQ ID NO:2 (AQGV), connected to the antibody-like molecule through a peptide bond, alternatively the antibody-like molecule is otherwise conjugated to the source of autophagy inhibiting amino acids. Conjugation methods are known in the art. Likewise, the source of autophagy inhibiting amino acids is a lipid vesicle such as a liposome, in particular, wherein the liposome comprises a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group SEQ ID NO: 1 (LQGV) and SEQ ID NO:2 (AQGV), preferably selected from the group AQ, LQ, GQ, AQL, LQL, GQL, PLQ, LQG, AQG

The disclosure provides a synthetic glutamine peptide that has been provided with an elastin-receptor binding motif and also provided (enriched) with selected amino acids that preferentially inhibit (mTOR mediated) autophagy of a cell after the peptide is hydrolyzed into its individual amino acid components in the lysosome of the cell. Inhibiting autophagy, by these selected autophagy inhibiting amino acids modulates the activity of immune cells; inhibiting autophagy, by these selected autophagy inhibiting amino acids modulates the permeability of vascular. These actions, alone or combined, contribute to the immune and/or vascular cells showing curative tissue repair activities after having been targeted with a (herein also called Q-ER) peptide according to the disclosure. The disclosure provides a curative and tissue repair supporting Q-ER peptide, the peptide comprising a synthetic peptide or functional analogue thereof, provided with a glutamine (Q) and with an elastin-receptor (ER) binding amino acid sequence motif and also comprising at least 50% amino acids selected from the group of autophagy inhibiting amino acids alanine (in one letter code: A), glutamine (Q), glycine (G), valine (V), leucine (L), proline (P), and arginine (R).

In a preferred embodiment, the disclosure provides a Q-ER peptide or functional analogue, that has been provided with an elastin-receptor binding motif and also comprises at least 60%, more preferably at least 75%, most preferably at least 90% amino acids selected from the group of autophagy inhibiting amino acids alanine (in one letter code: A), glutamine (Q), glycine (G), valine (V), leucine (L), proline (P), and arginine (R). Such a peptide is herein also called a Q-ER peptide, Q standing for glutamine, ER standing for elastin receptor. In a first embodiment, a Q-ER peptide, comprising or consisting of a synthetic peptide or functional analogue thereof, is provided with at least one elastin receptor binding amino acid motif, such as a PG-domain amino acid motif xGxxPG or xGxPGx or functional equivalent thereof, the PG-domain motif allowing targeting of the peptide to the elastin receptor complex (ER), wherein at least one amino acid at position x is selected from the group of amino acids alanine (in one letter code: A), glutamine (Q), glycine (G), valine (V), leucine (L), isoleucine (I), proline (P) and arginine, the peptide provided with at least one glutamine (Q).

In a preferred embodiment, the disclosure also provides a peptide (herein also termed Q-ER peptide) comprising at least 7 amino acids and at most 30 amino acids comprising a sequence of the formula ϕn xGxxPG, or xGxxPG ϕn, or ϕn xGxxPG ϕm wherein x is a naturally occurring amino acid, ϕ is an autophagy inhibiting amino acid and n = an integer from 1 to 24 and m is an integer from 1-23, whereby n+m is no greater than 24. In a preferred embodiment, n+m is no greater than 16, more preferably no greater than 12, more preferably no greater than 8. In a preferred embodiment, the disclosure provides a Q-ER peptide according to the disclosure wherein ϕn and/or ϕm comprise a dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group SEQ ID NO: 1 (LQGV) and SEQ ID NO:2 (AQGV), preferably selected from the group AQ, LQ, GQ, AQL, LQL, GQL, PLQ, LQG, AQG

The disclosure also provides a pharmaceutical formulation comprising a peptide (herein also termed Q-ER peptide) comprising at least 7 amino acids and at most 30 amino acids comprising a sequence of the formula ϕn xGxxPG, or xGxxPG ϕn, or ϕn xGxxPG ϕm wherein x is a naturally occurring amino acid, ϕ is an autophagy inhibiting amino acid and n = an integer from 1 to 24 and m is an integer from 1-23, whereby n+m is no greater than 24. In a preferred embodiment, n+m is no greater than 16, more preferably no greater than 12, more preferably no greater than 8. In a preferred embodiment the disclosure provides a pharmaceutical formulation comprising a peptide comprising xGxxPG and a peptide selected from dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group SEQ ID NO: 1 (LQGV) and SEQ ID NO:2 (AQGV), for use in lowering autophagy, wherein x represents a naturally occurring amino acid and at least one pharmaceutically acceptable excipient.

The disclosure also provides a pharmaceutical formulation comprising a peptide (herein also termed Q-ER peptide) comprising at least 7 amino acids and at most 30 amino acids comprising a sequence of the formula ϕn xGxxPG, or xGxxPG ϕn, or ϕn xGxxPG ϕm wherein x is a naturally occurring amino acid, ϕ is an autophagy inhibiting amino acid and n = an integer from 1 to 24 and m is an integer from 1-23, whereby n+m is no greater than 24. In a preferred embodiment, n+m is no greater than 16, more preferably no greater than 12, more preferably no greater than 8and at least one pharmaceutically acceptable excipient. In a preferred embodiment the disclosure provides a pharmaceutical formulation comprising a peptide comprising xGxxPG and a peptide selected from dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group SEQ ID NO: 1 (LQGV) and SEQ ID NO:2 (AQGV), for use in modifying vascular permeability, wherein x represents a naturally occurring amino acid and at least one pharmaceutically acceptable excipient.

The disclosure also provides a pharmaceutical formulation comprising a peptide (herein also termed Q-ER peptide) comprising at least 7 amino acids and at most 30 amino acids comprising a sequence of the formula ϕn xGxxPG, or xGxxPG ϕn, or ϕn xGxxPG ϕm wherein x is a naturally occurring amino acid, ϕ is an autophagy inhibiting amino acid and n = an integer from 1 to 24 and m is an integer from 1-23, whereby n+m is no greater than 24. In a preferred embodiment, n+m is no greater than 16, more preferably no greater than 12, more preferably no greater than 8and at least one pharmaceutically acceptable excipient. In a preferred embodiment the disclosure provides a pharmaceutical formulation comprising a peptide comprising xGxxPG and a peptide selected from dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group SEQ ID NO: 1 (LQGV) and SEQ ID NO:2 (AQGV), for use in modulating an immune response, wherein x represents a naturally occurring amino acid and at least one pharmaceutically acceptable excipient.

The disclosure also provides a pharmaceutical formulation comprising a peptide (herein also termed Q-ER peptide) comprising at least 7 amino acids and at most 30 amino acids comprising a sequence of the formula ϕn xGxxPG, or xGxxPG ϕn, or ϕn xGxxPG ϕm wherein x is a naturally occurring amino acid, ϕ is an autophagy inhibiting amino acid and n = an integer from 1 to 24 and m is an integer from 1-23, whereby n+m is no greater than 24. In a preferred embodiment, n+m is no greater than 16, more preferably no greater than 12, more preferably no greater than 8 and at least one pharmaceutically acceptable excipient. In a preferred embodiment the disclosure provides a pharmaceutical formulation comprising a peptide comprising xGxxPG and a peptide selected from dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group SEQ ID NO: 1 (LQGV) and SEQ ID NO:2 (AQGV), for use in improving or promoting tissue repair, wherein x represents a naturally occurring amino acid and at least one pharmaceutically acceptable excipient.

The disclosure also provides a pharmaceutical formulation comprising a peptide (herein also termed Q-ER peptide) comprising at least 7 amino acids and at most 30 amino acids comprising a sequence of the formula ϕn xGxxPG, or xGxxPG ϕn, or ϕn xGxxPG ϕm wherein x is a naturally occurring amino acid, ϕ is an autophagy inhibiting amino acid and n = an integer from 1 to 24 and m is an integer from 1-23, whereby n+m is no greater than 24. In a preferred embodiment, n+m is no greater than 16, more preferably no greater than 12, more preferably no greater than 8, further comprising insulin, preferably for use in the treatment of impairment of pancreatic beta-cell function. In a preferred embodiment, the disclosure provides a pharmaceutical formulation comprising a peptide comprising xGxxPG and a peptide selected from dipeptide selected from the group AQ, LQ, PQ, VQ, GQ, a tripeptide selected from the group AQL, LQL, PQL, VQL, GQL, PLQ, LQG, PQV, VGQ, LQP, LQV, AQG, QPL, PQV, VGQ, GQG or a tetrapeptide selected from the group SEQ ID NO: 1 (LQGV) and SEQ ID NO:2 (AQGV), for use in lowering autophagy, wherein x represents a naturally occurring amino acid, further comprising insulin, preferably for use in the treatment of impairment of pancreatic beta-cell function.

Typically the pharmaceutical formulations of the disclosure are intended for parenteral administration. In animal studies and clinical studies safe and efficacious doses can be established according to dose finding protocols well known to the skilled person. Typically peptides according to the disclosure without any targeting means need to be provided at high doses because of the limited half-life of oligopeptides in circulation. It is one of the advantages of the disclosure that by targeting less random circulation will occur and that by targeting more amino acids of the disclosure will be delivered where needed and therefore doses may be lower than of the peptides without targeting.

The disclosure further relates to the improvement of peptides comprising glutamine (Q), allowing efficient targeting of the glutamine-containing peptide (herein also termed glutamine peptide) to cells where the Q-ER peptide can exert its effects, therewith improving dosing requirements. Such a Q-ER peptide as provided by the disclosure is useful in methods and pharmaceutical compositions for the treatment of inflammatory and vascular conditions. The disclosure provides a synthetic Q-ER peptide of at most 30 amino acids, preferably at most 20 amino acids, more preferably at most 15 amino acids, most preferably at most 9 amino acids, the Q-ER peptide provided with at least one PG-domain motif GxxPG or GxPGx, the motif allowing targeting of the Q-ER peptide to the human elastin receptor complex (ER). Functional analogues of a Q-ER peptide may be selected from peptides comprising amino acids selected from the group of amino acids alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). In a preferred embodiment, the disclosure provides for a Q-ER peptide or functional analogue, that comprises at least 50%, more preferably at least 75%, most preferably at least 100% amino acids selected from the group of autophagy inhibiting amino acids alanine (in one letter code: A), glutamine (Q), glycine (G), valine (V), leucine (L), proline (P), and arginine (R). In a more preferred embodiment, the disclosure provides for a Q-ER peptide functional analogue, that comprises at least 50%, more preferably at least 75%, most preferably at least 100% amino acids selected from the group of autophagy inhibiting amino acids alanine (in one letter code: A), glutamine (Q), glycine (G), valine (V), leucine (L), and proline (P). In a most preferred embodiment, the disclosure provides for a Q-ER peptide functional analogue, that comprises at least 50%, more preferably at least 75%, most preferably at least 100% amino acids selected from the group of autophagy inhibiting amino acids alanine (in one letter code: A), glutamine (Q), glycine (G), leucine (L), and proline (P), ; these amino acids, and, in particular, glutamine (Q) and leucine (L), were shown to be most prominently capable inhibiting mTOR mediated autophagy, mTOR being an important switch governing proteogenesis and proteolysis (autophagy) in a cell.

Preferably, a functional analogue of the Q-ER peptide has a length in the range of 4-12 amino acids, more preferably 6-12 amino acids. Preferably, such a functional analogue is a linear peptide. A functional Q-ER peptide analogue according to the disclosure may be more preferably selected from the group consisting of peptides comprising a dipeptide sequence selected from the group of AQ, LQ, PQ, VQ, GQ. A functional Q-ER peptide analogue according to the disclosure may be more preferably selected from the group consisting of peptides comprising a tripeptide sequence selected from the group of AQL, LQL, PQL, VQL, GQL, PLQ, LQG, PQV, VGQ, LQP, LQV, AQG, QPL, PQV, VGQ, GQG. Preferably, a functional analogue is selected from the group of peptides having a functional elastin receptor binding motif, preferably with a PG-domain allowing for a type VIII beta-turn, preferably the PG-domain comprising a peptide sequence GxP or GxxP, more preferably comprising a peptide sequence GxPG or GxxPG, most preferably GxxPG.

It is preferred that at least one amino acid at position x is selected from the group of autophagy-inhibiting amino acids alanine, leucine, valine or glutamine (of these four, leucine and alanine are most preferred for inclusion in a Q-ER peptide with a PG-domain according to the disclosure), the peptide also provided with or containing at least one glutamine. In a preferred embodiment, a Q-ER peptide according to the disclosure comprises at least one amino acid sequence selected from the group of AQ, LQ, GQ, VQ, IQ, CQ, AQL, LQL, PQL, VQL, GQL, PLQ, LQG, PQV, VGQ, LQP, LQV, AQG, QPL, PQV, VGQ, GQG, SEQ ID NO:2 (AQGV) and SEQ ID NO: 1 (LQGV). In a Q-ER peptide for human use, the presence of the PG-domain motif GxxPG and inclusion of at least one amino acid sequence with SEQ ID NO: 1 (LQGV) and/or SEQ ID NO:2 (AQGV) is most preferred.

Herewith, the disclosure provides improved synthetic Q-ER peptides for use in the treatment of a diabetic, inflammatory or vascular condition, preferably for the treatment of such a condition in a human, the Q-ER peptides having been provided with a key motif of amino acids (PG-domain) allowing targeting to and docking of the improved Q-ER peptide with cells carrying the human elastin receptor complex, a receptor complex involved in modulating immune cell reactivity and/or vascular cell repair. The beneficial anti-diabetic, anti-inflammatory and vascular repair effect of the Q-ER peptide, once it has entered the target cell, is thought to be generated by inhibition of autophagy of the target cell through autophagy-inhibiting-amino acids generated by hydrolysis of the Q-ER peptide and act on the mammalian target of rapamycin (mTOR) complex.

A preferred autophagy-inhibiting amino acid included in a Q-ER peptide as provided by the disclosure is selected from the group of amino acids alanine (A), proline (P), leucine (L) and glutamine (QMost preferred autophagy inhibiting amino acids for inclusion in a PG-domain comprising Q-ER peptide according to the disclosure are L-leucine, L-glutamine and L-alanine.

Inhibition of autophagy in the target cells (cells with an active elastin receptor complex such as immune cells or vascular cells are preferably targeted) by a Q-ER peptide according to the disclosure generally results in improved resistance to permeability and improved proliferation of vascular cells and reduced acute inflammatory activity and reduced extravasation of immune cells. Acute systemic conditions such as sepsis or systemic inflammatory response syndrome (SIRS), as well as chronic systemic vasculopathies in patients with a relative or absolute lack of C-peptide (as typically seen in type 1 diabetes and end-phase type 2 diabetes) often lead to a pathogenesis of micro-vascular damage involving detrimental activation and extravasation of immune cells (e.g., neutrophils, macrophages and lymphocytes) and destruction of the vascular cells (e.g., vascular endothelial cells, smooth muscle cells and fibroblasts), that form blood vessels. Targeting a Q-ER peptide according to the disclosure to these cells where it is then hydrolyzed and inhibits autophagy through the action of its individual amino acids inhibiting autophagy allows reduction of these pathogenic events with beneficial effects to the treatment of a patient suffering from the diabetic, conditions often seen due to lack of C-peptide, and seen with other (micro-)vascular and/or inflammatory conditions.

The human elastin receptor complex is typically activated by a peptide carrying a key motif of amino acids (PG-domain) found in elastin and in breakdown products (PG-domain-fragments) thereof. Herein a peptide or peptide fragment having a functional PG-domain is defined as a peptide having at least one GxxPx or GxPxx amino acid sequence motif, G being Glycine, P being Proline, x being any amino acid, the amino acid following P allowing for a type VIII-beta turn, a condition, which is considered always met when P is C-terminally followed by a G. The disclosure also provides a method for preventing or treating disease comprising providing a human with such a peptide capable of binding to (docking) and modulating an elastin receptor. In a further embodiment, the disclosure provides a synthetic peptide provided with at least one PG-domain pentapeptide motif GxxPG (G being glycine, P being proline, and x any amino acid), preferably wherein at least one amino acid at one position x is selected from the group of alanine, leucine, valine or isoleucine, the peptide always provided with at least one glutamine. The disclosure also provides synthetic peptides wherein this pentapeptide motif has been repeated at least once, optionally the PG-domain repeats are separated by a linker, such a linker may comprise one or more amino acids, such as one or more amino acids selected from the group of alanine, leucine, glycine, valine, isoleucine or glutamine.

The disclosure also provides a synthetic peptide provided with at least one hexapeptide motif xGxxPG wherein at least one amino acid at position x, more preferably at least at two positions x, is selected from the group of alanine, leucine, valine or isoleucine, the peptide always provided with at least one glutamine. The disclosure also provides synthetic peptides wherein this hexapeptide motif has been repeated at least once, optionally the repeats are separated by a linker, such a linker may comprise one or more amino acids, such as one or more amino acids selected from the group of alanine, leucine, glycine, valine, isoleucine or glutamine.

In particular, the disclosure provides a Q-ER peptide comprising at least one amino acid sequence selected from the group of known glutamine peptides capable of treating systemic conditions AQ, LQ, GQ, VQ, PQ, IQ, CQ, AQG, LQG, SEQ ID NO:2 (AQGV) and SEQ ID NO: 1 (LQGV); by inclusion of the pentapeptide motif GxxPG recognised by inflammatory and vascular cells carrying the elastin receptor complex, the PG-domain allowing the peptides of the disclosure to be targeted to the cells that are typically involved in vascular and/or inflammatory conditions, which then benefit from the amino acid sequence selected from the group of known peptides capable of treating such conditions. In a preferred embodiment, the disclosure provides a hexa-, hepta- or octa-, nona-, deca, undeca or dodecapeptide according to the disclosure. Hexa-, hepta- or octa-, nona-peptides are most preferred, larger peptides generally being too immunogenic for repeated application over long time. In a preferred embodiment the disclosure provides a synthetic Q-ER peptide provided with at least one PG-domain pentapeptide motif GxxPG and having at least one amino acid sequence with SEQ ID NO: 1 (LQGV) and/or SEQ ID NO:2 (AQGV), in a preferred embodiment such a peptide having an amino acid sequence with SEQ ID NO:1 (LQGV) and/or SEQ ID NO:2 (AQGV) is preferably selected from the group SEQ ID NO:5 (AQGVAPG), SEQ ID NO:6 (LQGVAPG), SEQ ID NO:7 (AQGVLPG), SEQ ID NO:8 (LQGVLPG), SEQ ID NO:9 (AQGVAPGQ), SEQ ID NO: 10 (LQGVAPGQ), SEQ ID NO: 11 (AQGVLPGQ), and SEQ ID NO: 12 (LQGVLPGQ). The present specification provides a solution for the loss of active non-targeted glutamine peptide preparations by providing a synthetic Q-ER peptide provided with an elastin receptor complex (ERC) recognition PG-domain motif targeting the ERC. Typically, such motifs in humans, primates and most mammals preferably have a consensus hexapeptide sequence xGxxPG, some other mammals (e.g., ruminants) preferably have a consensus pentapeptide sequence xGxPG, which seems of functional equivalent binding affinity in some species. Preferably, with the synthetic Q-ER peptides disclosed herein, at least one of the undefined amino acid positions (x) stands for an aliphatic amino acid, such as valine (V), glutamine (Q) or, most preferably, leucine (L) or alanine (A). The disclosure provides a synthetic peptide having a hexapeptide xGxxPG motif, such as, for example, a peptide having hexapeptide motifs SEQ ID NO:3 (QGVLPG) or SEQ ID NO:4 (QGVAPG), wherein an N-terminal amino acid of the peptide is selected from valine (V), glycine (G), isoleucine (I) or, most preferably, leucine (L) or alanine (A). In another preferred embodiment, the disclosure provides a peptide for use in humans with a hexapeptide motif SEQ ID NO:3 (QGVLPG) or SEQ ID NO:4 (QGVAPG) wherein the N-terminal amino acid is alanine (A) or preferably leucine (L), such as synthetic heptapeptide SEQ ID NO:5 (AQGVAPG), SEQ ID NO:6 (LQGVAPG), SEQ ID NO:7 (AQGVLPG) or SEQ ID NO:8 (LQGVLPG), respectively. The disclosure also provides a synthetic Q-ER peptide wherein one of these PG-domain motifs has been repeated at least once. Optionally the repeats are separated by a linker, such a linker may comprise one or more amino acids, such as one or more amino acids selected from the group of glycine, alanine, leucine, valine, isoleucine or glutamine. A non-glutamine N-terminal amino acid preferably is attached that protects the glutamine from transition to pyro-glutamine. In a further embodiment, again preferred for human use, the disclosure provides a synthetic peptide having a xGxxPG motif, such as, for example, SEQ ID NO:3 (QGVLPG) or SEQ ID NO:4 (QGVAPG), wherein the N-terminal amino acid is selected from as valine (V), glycine (G), isoleucine (I) or, most preferably, leucine (L) or alanine (A). In preferred embodiment, the disclosure provides peptide for use in humans with a hexapeptide motif SEQ ID NO:3 (QGVLPG) or SEQ ID NO:4 (QGVAPG) wherein the N-terminal amino acid is alanine (A) or preferably leucine (L), and the C-terminal amino acid is glutamine (Q), , the N-terminal amino acid protecting the glutamine from transition to pyro-glutamine, and the C-terminal amino acid providing an extra glutamine being targeted to cells having the xGxxPG recognizing receptor, the elastin receptor complex (ERC). Typical examples as provided herein are synthetic octapeptide SEQ ID NO:9 (AQGVAPGQ), SEQ ID NO: 10 (LQGVAPGQ), SEQ ID NO: 11 (AQGVLPGQ) or SEQ ID NO: 12 (LQGVLPGQ). In another preferred embodiment, the disclosure provides a peptide for use in humans with a hexapeptide motif SEQ ID NO: 13 (LQGQAPG) or SEQ ID NO: 14 (QGQAPG) wherein the N-terminal amino acid is alanine (A) or preferably leucine (L), and the C-terminal amino acid is glutamine (Q), the N-terminal amino acid protecting the glutamine from transition to pyro-glutamine, and the C-terminal amino acid providing an extra glutamine being targeted to cells having the xGxxPG recognizing receptor, the elastin receptor complex (ERC), and having been provide with yet another, third glutamine to be targeted to the cell. Typical examples as provided herein are synthetic octapeptide SEQ ID NO: 15 (AQGQAPGQ), SEQ ID NO:16 (LQGQAPGQ), SEQ ID NO: 17 (AQGQLPGQ) or SEQ ID NO:18 (LQGQLPGQ). In a preferred embodiment, the disclosure provides a Q-ER peptide according to the disclosure having a GxxPG elastin-receptor-binding motif and amino acids GQ at position xx such as identified in table 3. A preferred Q-ER peptide is selected from the group comprising SEQ ID NO:46 (VQGGQPGQ), SEQ ID NO:49 (AQGGQPGQ) and SEQ ID NO:53 (GQGGQPGQ).

In a preferred embodiment, the disclosure provides a Q-ER peptide according to the disclosure having a GxxPG elastin-receptor-binding motif and amino acids VQ at position xx such as identified in table 4. A much preferred Q-ER peptide is selected from the group comprising SEQ ID NO:51 (GQGVQPGQ), SEQ ID NO:44 (VQGVQPGQ), SEQ ID NO:24 (LQGVQPGQ), SEQ ID NO:22 (AQGVQPG) and SEQ ID NO:25 (AQGVQPGQ).

In a preferred embodiment, the disclosure provides a Q-ER peptide according to the disclosure having a GxxPG elastin-receptor-binding motif and amino acids VA at position xx as identified in table 5. A much preferred Q-ER peptide is selected from the group comprising SEQ ID NO:4 (QGVAPG), SEQ ID NO:5 (AQGVAPG), SEQ ID NO:6 (LQGVAPG), SEQ ID NO:9 (AQGVAPGQ), SEQ ID NO:10 (LQGVAPGQ), SEQ ID NO:27 (VQGVAPG), SEQ ID NO:29 (GQGVAPG), SEQ ID NO:33 (VQGVAPGQ) and SEQ ID NO:37 (GQGVAPGQ).

In a preferred embodiment, the disclosure provides a Q-ER peptide according to the disclosure having a GxxPG elastin-receptor-binding motif and amino acids VL at position xx as identified in table 6. A much preferred Q-ER peptide is selected from the group comprising SEQ ID NO:3 (QGVLPG), SEQ ID NO:7 (AQGVLPG), EQ ID NO:8 (LQGVLPG), SEQ ID NO: 11 (AQGVLPGQ), SEQ ID NO: 12 (LQGVLPGQ), SEQ ID NO:26 (VQGVLPG), SEQ ID NO:28 (GQGVLPG), SEQ ID NO:32 (VQGVLPGQ) and SEQ ID NO:36 (GQGVLPGQ).

In a preferred embodiment, the disclosure provides a Q-ER peptide according to the disclosure having a GxxPG elastin-receptor-binding motif and amino acids QA at position xx as identified in table 7. A much preferred Q-ER peptide is selected from the group comprising SEQ ID NO: 13 (LQGQAPG), SEQ ID NO: 14 (QGQAPG), SEQ ID NO: 15 (AQGQAPGQ) and SEQ ID NO: 16 (LQGQAPGQ).

In a preferred embodiment, the disclosure provides a Q-ER peptide according to the disclosure having a GxxPG elastin-receptor-binding motif and amino acids AQ at position xx as identified in table 8. A much preferred Q-ER peptide is selected from the group comprising SEQ ID NO:45 (VQGAQPGQ), SEQ ID NO:41 (LQGAQPGQ), SEQ ID NO:48 (AQGAQPGQ) and SEQ ID NO:52 (GQGAQPGQ).

In a preferred embodiment, the disclosure provides a Q-ER peptide according to the disclosure having a GxxPG elastin-receptor-binding motif and amino acids QL at position xx as identified in table 9. A much preferred Q-ER peptide is selected from the group comprising SEQ ID NO:17 (AQGQLPGQ), SEQ ID NO:18 (LQGQLPGQ), SEQ ID NO:21 (AQPGQLPG), SEQ ID NO:30 (LQGQLPG), SEQ ID NO:31 (VQGQLPG), SEQ ID NO:34 (AQGQLPG), SEQ ID NO:35 (GQGQLPG), SEQ ID NO:38 (VQGQLPGQ) and SEQ ID NO:39 (GQGQLPGQ).

In a preferred embodiment, the disclosure provides a Q-ER peptide according to the disclosure having a GxxPG elastin-receptor-binding motif and amino acids LQ at position xx as identified in table 10. A much preferred Q-ER peptide is selected from the group comprising SEQ ID NO:20 (LQVGLQPG), SEQ ID NO:40 (LQGLQPGA), SEQ ID NO:42 (LQGLQPGQ), SEQ ID NO:43 (VQGLQPGQ), SEQ ID NO:47 (AQGLQPGQ) and

SEQ ID NO:50 (GQGLQPGQ).

In a most preferred embodiment, the disclosure provides a Q-ER peptide according to the disclosure having a GxxPG elastin-receptor-binding motif and is furthermore provided with a peptide sequence with SEQ ID NO:1 (LQGV) as identified in table 11. A most preferred Q-ER peptide is selected from the group comprising SEQ ID NO:6 (LQGVAPG), SEQ ID NO:8 (LQGVLPG), SEQ ID NO:10 (LQGVAPGQ), SEQ ID NO:12 (LQGVLPGQ) and SEQ ID NO:24 (LQGVQPGQ).

In a most preferred embodiment, the disclosure provides a Q-ER peptide according to the disclosure having a GxxPG elastin-receptor-binding motif and is furthermore provided with a peptide sequence with SEQ ID NO:2 (AQGV) as identified in table 12. A most preferred Q-ER peptide is selected from the group comprising SEQ ID NO:5 (AQGVAPG), SEQ ID NO:7 (AQGVLPG), SEQ ID NO:9 (AQGVAPGQ), SEQ ID NO:11 (AQGVLPGQ), SEQ ID NO:22 (AQGVQPG) and SEQ ID NO:25 (AQGVQPGQ).

Additional useful synthetic Q-ER peptides having been provided with a PG-domain binding motif are listed in the detailed description or elsewhere herein (in particular, Q-Vh and/or Q-Vhh).

The peptides as provided herein are useful in the treatment of acute conditions, such as acute kidney injury, also in acute systemic inflammatory conditions such as sepsis or systemic inflammatory response syndrome (SIRS), leading to vascular damage and often aggravated by (multiple organ) organ failure, or inflammatory conditions. The peptides of the disclosure are particularly useful in vascular conditions accompanying diabetes due to reduced beta-cell activity (as in type 1 diabetes and in end-stage type 2 diabetes), as such patients show reduced C-peptide and insulin levels and therewith generally suffer from excess (micro) vascular permeability and excess leucocyte extravasation, together with excess circulating blood glucose. It is preferred that such subjects, when given or treated with Q-ER-peptide receive accompanying or simultaneous treatment with an anti-diabetic composition such as insulin to remedy risen glucose levels as well. In a further embodiment of the disclosure, synthetic Q-ER peptides as provided herein are encapsulated in an acid resistant capsule. Such (pharmaceutical) capsules are widely used in the pharmaceutical field as oral dosage forms for administration to humans and animals. Filled with a Q-ER peptide according to the disclosure, such a capsule is useful for the enteral administration of a synthetic Q-ER peptide provided with at least one, preferably two or three PG-domain motifs GxxPG (G being glycine, P being proline, and x any amino acid), preferably wherein at least one amino acid at one position x is selected from the group of alanine, leucine, valine or isoleucine, the peptide also provided with at least one glutamine. Such administration would, for example, alleviate or treat diseases such as Crohn’s disease in which gut endothelial cells need regeneration. Also, such administration would be useful in treating gastro-intestinal damage obtained after excess radiation. The Q-ER peptides provided herein may also be advantageously combined with other therapeutics such as immunomodulatory substances, e.g., with (immunomodulatory) peptides, in particular, peptides such as peptides with SEQ ID NO: 1 (LQGV), AQG, or SEQ ID NO:2 (AQGV), or with conventionalimmunomodulators, such as with immunomodulatory antibodies or proteins acting against cytokines as TNF-alpha, IL-1 or IL-6, or corticosteroid formulations.

The disclosure also provides a method for treatment of an acute and/or systemic condition of a subject suffering or believed to be suffering from the condition the method comprising providing the subject, preferably parenterally, intravenously or intraperitoneally with a Q-ER peptide according to the disclosure, preferably a synthetic Q-ER peptide, of at most 30 amino acids, the Q-ER peptide provided with at least one PG-domain motif GxxPG allowing targeting of the peptide to the elastin receptor complex, wherein at least one amino acid at position x is selected from the group of alanine, leucine, valine or isoleucine, the peptide also provided with at least one glutamine. It is preferred that the Q-ER peptide comprises at least one amino acid sequence selected from the group of AQ, LQ, GQ, VQ, IQ, CQ, AQG, LQG, SEQ ID NO:2 (AQGV) and SEQ ID NO: 1 (LQGV). In a method of treatment of a human subject as provided herein according to the disclosure, treating the subject with a Q-ER peptide having the PG-domain motif GxxPG and having at least one amino acid sequence with SEQ ID NO: 1 (LQGV) and/or SEQ ID NO:2 (AQGV) is most preferred.

The disclosure also provides a method for treatment of an vascular and/or inflammatory condition of a human subject suffering or believed to be suffering from the condition the method comprising providing the subject, preferably parenterally, intravenously or intraperitoneally with a hepta-, octa-, nona, deca, undeca- or dodeca-peptide, most preferably a hepta-, octa-, nona-peptide, provided with at least one PG-domain motif GxxPG allowing targeting of the peptide to the elastin receptor complex, wherein at least one amino acid at position x is selected from the group of alanine, leucine, valine or isoleucine, the peptide also provided with at least one glutamine, according to the disclosure. A method is preferred wherein the peptide according to the disclosure is provided with at least two glutamines, more preferably three glutamines.

The disclosure also provides a method for treatment of an inflammatory condition of a subject suffering or believed to be suffering from the condition the method comprising providing the subject, preferably parenterally, intravenously or intraperitoneally, with a peptide having at least one amino acid sequence with SEQ ID NO:1 (LQGV) and/or SEQ ID NO:2 (AQGV) according to the disclosure, preferably a synthetic peptide selected from the group SEQ ID NO:5 (AQGVAPG), SEQ ID NO:6 (LQGVAPG), SEQ ID NO:7 (AQGVLPG) and SEQ ID NO:8 (LQGVLPG).

The disclosure also provides a method for treatment of an vascular and/or inflammatory condition of a subject suffering or believed to be suffering from the condition the method comprising providing the subject, preferably parenterally, intravenously or intraperitoneally, with a peptide having at least one amino acid sequence with SEQ ID NO: 1 (LQGV) and/or SEQ ID NO:2 (AQGV) according to the disclosure, preferably a synthetic peptide selected from the group SEQ ID NO:9 (AQGVAPGQ), SEQ ID NO:10 (LQGVAPGQ), SEQ ID NO: 11 (AQGVLPGQ) and SEQ ID NO: 12 (LQGVLPGQ).

The disclosure also provides a method for treatment of an vascular and/or inflammatory condition of a subject suffering or believed to be suffering from the condition, the method comprising providing the subject, preferably parenterally, intravenously or intraperitoneally, with a peptide having at least one amino acid sequence with SEQ ID NO: 1 (LQGV) and/or SEQ ID NO:2 (AQGV) according to the disclosure, preferably a synthetic peptide selected from the group SEQ ID NO:15 (AQGQAPGQ), SEQ ID NO:16 (LQGQAPGQ), SEQ ID NO: 17 (AQGQLPGQ) and SEQ ID NO: 18 (LQGQLPGQ).

### DETAILED DESCRIPTION

Functional analogues of a Q-ER peptide may be selected from peptides comprising amino acids selected from the group of amino acids alanine (in one letter code: A), glutamine (Q), glycine (G), valine (V), leucine (L), isoleucine (I), proline (P) and arginine (R), more preferably selected from the group leucine (L), alanine (A), glutamine (Q), glycine (G) and proline (P). In a preferred embodiment, the disclosure provides for a Q-ER peptide or functional analogue, that comprises at least 50%, more preferably at least 75%, most preferably at least 100% amino acids selected from the group of autophagy inhibiting amino acids alanine (in one letter code: A), glutamine (Q), glycine (G), valine (V), leucine (L), proline (P), and arginine (R). In a more preferred embodiment, the disclosure provides for a Q-ER peptide functional analogue, that comprises at least 50%, more preferably at least 75%, most preferably at least 100% amino acids selected from the group of autophagy inhibiting amino acids alanine (in one letter code: A), glutamine (Q), glycine (G), valine (V), leucine (L), and proline (P). In a most preferred embodiment, the disclosure provides for a Q-ER peptide functional analogue, that comprises at least 50%, more preferably at least 75%, most preferably at least 100% amino acids selected from the group of autophagy inhibiting amino acids alanine (in one letter code: A), glutamine (Q), glycine (G), leucine (L), and proline (P). Preferably, a functional analogue of the Q-ER peptide has a length in the range of 4-12 amino acids, more preferably 6-12 amino acids. Preferably, such a functional analogue is a linear peptide. A functional Q-ER peptide analogue according to the disclosure may be more preferably selected from the group consisting of peptides comprising a dipeptide sequence selected from the group of AQ, LQ, PQ, VQ, GQ. A functional Q-ER peptide analogue according to the disclosure may be more preferably selected from the group consisting of peptides comprising a tripeptide sequence selected from the group of AQL, LQL, PQL, VQL, GQL, PLQ, LQG, PQV, VGQ, LQP, LQV, AQG, QPL, PQV, VGQ, GQG. Preferably, a functional analogue is selected from the group of synthetic peptides provided with or having a functional elastin receptor binding motif, preferably with a PG-domain allowing for a type VIII beta-turn, preferably the PG-domain comprising a peptide sequence GxP or GxxP, more preferably comprising a peptide sequence GxPG or GxxPG, most preferably GxxPG.

In another embodiment, a Q-ER peptide, or a functional analogue thereof, is provided for use in the treatment of a human subject having impaired beta-cell function. In a further embodiment, the impaired beta-cell function is in diabetes. In one embodiment, an Q-ER peptide, or a functional analogue thereof, is provided for use in the treatment of a human subject for improving beta-cell function. Beta-cell function can be assessed by determining pre-proinsulin production by beta-cells of the pancreas, preferably by assessing insulin and/or C-peptide in blood, serum or plasma. Defective function of the pancreatic β-cells is now accepted to be a hallmark of type 1 and end-phase-type 2 diabetes. The importance of the β-cells in type 1 diabetes has long been accepted. On the contrary, the necessity of a pancreatic defect in end-phase type 2 diabetes has only recently been widely appreciated. In clinical practice, fasting samples or simple stimulation tests, such as the OGTT, the glucagon stimulatory test, or the standard breakfast test, which are the most widely used and validated, may be used as parameters of insulin secretion. In these tests, mathematical handling of fasting levels of glucose, insulin, and C-peptide concentrations is the simplest method. Mathematical handling of the fasting levels has been used for the calculation of insulin sensitivity and secretion by introducing various indices, such as HOMA, fasting beta-cell responsiveness (M0), and QUICKI. Assessing beta-cell function in humans is standard clinical practice (e.g., by determining glucose levels, insulin or C-peptide). Improvements in beta-cell function as compared with not receiving the Q-ER peptide can include progressing to a beta-cell function stage as assessed by methods such as HOMA, fasting beta-cell responsiveness (M0), and QUICKI. Improvements in beta-cell function also include having an improvement in glucose levels. Irrespective of what assessment is made, the use of the Q-ER peptide, or analogue thereof, can improve beta-cell function in humans having beta-cell failure and/or an impairment of beta-cell function in subjects.

Not only does the use of the Q-ER peptide allow for improving beta-cell function it can also prevent a reduction and/or an impairment of beta-cell function. Accordingly, diabetes may be prevented. Preferably, in prevention of a human subject having impaired beta-cell function, the Q-ER peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the Q-ER peptide is administered for at least 1 hour, more preferably at least 1.5 hours, most preferably at least 2 hours. Preferably, the administration of the Q-ER peptide is at a rate of at least 10 mg/kg/hr and administered for at least 1 hour, more preferably at least 1.5 hours, most preferably at least 2 hours, such as at least 2.5 hours. If required, Q-ER peptide may be administered together with an insulin or insulin analogue. Hence, in one embodiment, the use of the Q-ER peptide, or analogue thereof, allows to maintain beta-cell function in human patients. In another embodiment, the use of the Q-ER peptide, or analogue thereof, allows to prevent a reduction and/or impairment of beta-cell function in human patients.

In another embodiment, a Q-ER peptide, or a functional analogue thereof, is provided for use in the treatment of a human subject having impaired kidney function. In a further embodiment, the impaired kidney function is acute kidney injury (AKI). In one embodiment, an Q-ER peptide, or a functional analogue thereof, is provided for use in the treatment of a human subject for improving kidney function. Kidney function can be assessed by determining the glomerular filtration rate (GFR), for example, by assessing the clearance of iohexol from blood plasma. Kidney function can also be assessed by measuring plasma levels of creatinine and calculating an estimated GFR (eGFR) function therefrom, also referred to as the MDRD formula or equation, taking into account patient characteristics such as sex, age and race (Modification of Diet in Renal Disease). Kidney function can be assessed based on GFR measurements (or estimates thereof based on MDRD) by applying the RIFLE criteria. Having a RIFLE score, which is in the stage of risk, injury, failure, loss or ESKD, can be indicative of kidney injury and/or impairment of kidney function. Assessing kidney function in humans is standard clinical practice (e.g., by determining GFR, creatinine clearance, and/or eGFR/MDRD). Improvements in kidney function as compared with not receiving the Q-ER peptide can include progressing to a kidney function stage as assessed under the RIFLE criteria to a less severe stage (e.g., a patient progressing from having injury to being at risk of injury or having no AKI). Improvements in kidney function also include having an improvement in GFR or eGFR scores. Irrespective of what assessment is made, the use of the Q-ER peptide, or analogue thereof, can improve kidney function in humans having kidney injury and/or an impairment of kidney function in subjects absent of immunomodulatory effects.

Not only does the use of the Q-ER peptide allow for improving kidney function it can also prevent a reduction and/or an impairment of kidney function. Accordingly, AKI may be prevented. Preferably, in prevention of a human subject having impaired kidney function, the Q-ER peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the Q-ER peptide is administered for at least 1 hour, more preferably at least 1.5 hours, most preferably at least 2 hours. Preferably, the administration of the Q-ER peptide is at a rate of at least 10 mg/kg/hr and administered for at least 1 hour, more preferably at least 1.5 hours, most preferably at least 2 hours, such as at least 2.5 hours. Hence, in one embodiment, the use of the Q-ER peptide, or analogue thereof, allows to maintain kidney function in human patients. In another embodiment, the use of the Q-ER peptide, or analogue thereof, allows to prevent a reduction and/or impairment of kidney function in human patients. For example, a human patient that may be classified as having no AKI, or being at risk of having kidney injury (such as AKI), when such a patient receives treatment with the Q-ER peptide, such a patient may maintain its status instead of progressing to a kidney function, which is a more severe stage. Hence, human patients that are at risk of developing kidney injury, e.g., due to (induced) trauma, such human patients as a result of receiving treatment with the Q-ER peptide, or analogue thereof, can maintain their kidney function status.

In another embodiment, the use of a Q-ER peptide, or a functional analogue thereof, reduces adverse fluid retention in the human subject. Fluid retention or fluid overload can occur in human subjects, symptoms of which e.g., include weight gain and edema. Fluid retention can be the result of reduced kidney function and/or diabetes type 1 or end-phase type 2 (when no or little endogenous C-peptide is produced by the subject and micro-vascular flow is compromised). Fluid retention can be the result of leaky capillaries. Hence, the use of Q-ER peptide, and analogues thereof, may have an effect on the leakiness of capillaries, reducing leakage of plasma and extravasation of immune cells (leucocytes) from the blood to peripheral tissue and/or organs. Most preferably, edema and/or leukocyte extravasation is reduced and/or avoided by the use of Q-ER peptide. Such may also be referred to as adverse fluid retention as it has an adverse effect on the patient. Whichever is the cause of fluid retention, the use of an Q-ER peptide, or a functional analogue thereof, can improve fluid retention (with or without extravasated leucocytes) in human subjects thereby alleviating symptoms associated with fluid retention such as weight gain and edema, which subsequently can reduce the use of diuretics. Preferably, in use of (in particular, the smaller) Q-ER peptides (5-15 amino acids) to improve fluid retention, the Q-ER peptide is administered at a rate which is at least 10 mg/kg patient weight per hour (mg/kg/hr). Preferably the administration rate is at least 20 mg, at least 30, at least 40 or, most preferably, at least 50 mg/kg/hr. Preferably, the Q-ER peptide is administered for at least 1 hour, more preferably at least 1.5 hours, most preferably at least 2 hours. Preferably, the administration of the Q-ER peptide is at a rate of at least 20 mg/kg/hr and administered for at least 1 hour, more preferably at least 1.5 hours, most preferably at least 2 hours, such as at least 2.5 hours.

In another embodiment, the use of the Q-ER peptide, or a functional analogue thereof, in accordance with the disclosure, is not restricted to patients having kidney injury, neither to patients having beta-cell failure. The use of a Q-ER peptide, or a functional analogue thereof, in accordance with the disclosure, includes the treatment of human patients that are believed to be at risk of having a systemic inflammation and/or are anticipated to require anti-inflammatory therapy. Such human patients include patients that are to be admitted, or are expected to be admitted, into intensive care. Hence, the use of the Q-ER peptide, or a functional analogue thereof, includes a use for induced trauma, such as surgery. Induced trauma includes any physical injury to the human body and typically can include the loss of blood and/or injury to tissues of the human subject. Induced trauma includes e.g., surgery. Hence, in a preferred embodiment, the induced trauma is surgery. The use of the Q-ER peptide for treatment of induced trauma, such as surgery, may be before, during and/or after surgery. It may be preferred that the use of the Q-ER peptide, or an analogue thereof, is during surgery. Preferably, the Q-ER peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the Q-ER peptide is administered for at least 1 hour, more preferably at least 1.5 hours, most preferably at least 2 hours. Preferably, the administration of the Q-ER peptide is at a rate of at least 20 mg/kg/hr and administered for at least 1 hour, more preferably at least 1.5 hours, most preferably at least 2 hours, such as at least 2.5 hours.

In another, or further, embodiment, the use of an Q-ER peptide, or a functional analogue thereof, for use in accordance with the disclosure is for use is in a human subject having heart failure. Preferably, in use in a human subject having heart failure, the Q-ER peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the Q-ER peptide is administered for at least 1 hour, more preferably at least 1.5 hours, most preferably at least 2 hours. Preferably, the administration of the Q-ER peptide is at a rate of at least 20 mg/kg/hr and administered for at least 1 hour, more preferably at least 1.5 hours, most preferably at least 2 hours, such as at least 2.5 hours.

The disclosure includes the use of an Q-ER peptide, or a functional analogue thereof, for use in the treatment of a human subject considered at risk or suffering from fluid overload, the use comprising modifying fluid retention in the human subject. The use of Q-ER peptide, or a functional analogue thereof, in accordance with the disclosure, includes the treatment of human patients that are believed to be at risk of having fluid overload and/or anticipated to require hemodynamic therapy. Such human patients include patients that are to be admitted, or are expected to be admitted, into intensive care. Hence, the use of Q-ER peptide, or a functional analogue thereof, includes a use for prevention of induced fluid overload, such as with fluid therapy. Preferably, in use for prevention of induced fluid overload, the Q-ER peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the Q-ER peptide is administered for at least 1 hour, more preferably at least 1.5 hours, most preferably at least 2 hours. Preferably, the administration of the Q-ER peptide is at a rate of at least 20 mg/kg/hr and administered for at least 1 hour, more preferably at least 1.5 hours, most preferably at least 2 hours, such as at least 2.5 hours. In another embodiment, the use of Q-ER peptide, or a functional analogue thereof, in accordance with the disclosure, is not restricted to patients having kidney injury and/or requiring hemodynamic therapy.

The disclosure includes the use of an Q-ER peptide, or a functional analogue thereof, for use in the treatment of a human subject to improve the subject’s length of stay at the ICU, further to shorten the subject’s length of stay at the ICU. One way in which this may be attained is by modifying fluid retention in the human subject. The use of Q-ER peptide, or a functional analogue thereof, in accordance with the disclosure, includes the treatment of human patients that are believed to be at risk from treatment with a vasopressor or an inotropic medication and/or anticipated to require hemodynamic therapy with fluid therapy. Such human patients include patients that are or are to be admitted, or are expected to be admitted, into intensive care, and for which shortening length-of-stay at ICU is desired. Hence, the use of Q-ER peptide, or a functional analogue thereof, includes a use for the treatment of human patients that are believed to be at risk from treatment with detrimental vasopressor or inotropic medication and/or with fluid therapy, is provided as shown e.g., in the examples. Preferably, in use for shortening a subject’s length of stay at the ICU, in human patients that are believed to be at risk, the Q-ER peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the Q-ER peptide is administered for at least 1 hour, more preferably at least 1.5 hours, most preferably at least 2 hours. Preferably, the administration of the Q-ER peptide is at a rate of at least 20 mg/kg/hr and administered for at least 1 hour, more preferably at least 1.5 hours, most preferably at least 2 hours, such as at least 2.5 hours.

In another embodiment, the use of Q-ER peptide, or a functional analogue thereof, in accordance with the disclosure, is not restricted to patients having kidney injury, beta-cell failure and/or requiring hemodynamic therapy. The disclosure includes the use of an Q-ER peptide, or a functional analogue thereof, for use in the treatment of a human subject to improve the subject’s length of stay at the hospital, further to shorten the subject’s length of stay at the hospital, the use comprising modifying fluid retention in the human subject. The use of Q-ER peptide, or a functional analogue thereof, in accordance with the disclosure, includes the treatment of human patients that are believed to be at risk from treatment with a vasopressor or an inotropic medication and/or anticipated to require hemodynamic therapy with fluid therapy. Such human patients include patients that are or are to be admitted, or are expected to be admitted, into intensive care or hospital, and for which shortening length-of-stay at hospital is desired. Hence, the use of Q-ER peptide, or a functional analogue thereof, includes a use for the treatment of human patients that are believed to be at risk from treatment with detrimental vasopressor or inotropic medication and/or with fluid therapy, is provided. Preferably, in use for shortening a subject’s length of stay at the ICU, in human patients that are believed to be at risk, the Q-ER peptide is administered at a rate that is at least 10 mg/kg patient weight per hour (mg/kg/hr). Preferably, the administration rate is at least 20 mg, at least 30, at least 40 or, most preferably, at least 50 mg/kg/hr. Preferably, the Q-ER peptide is administered for at least 1 hour, more preferably at least 1.5 hours, most preferably at least 2 hours. Preferably, the administration of the Q-ER peptide is at a rate of at least 20 mg/kg/hr and administered for at least 1 hour, more preferably at least 1.5 hours, most preferably at least 2 hours, such as at least 2.5 hours.

Preferably, the use of the Q-ER peptide, or a functional analogue thereof, in accordance with the disclosure and as described above, involves the administration of the peptide into the bloodstream. It is understood that administration into the bloodstream comprises e.g., intravenous administration or intra-arterial administration. A constant supply of Q-ER peptide, or an analogue thereof, is preferred, e.g., via an infusion wherein the Q-ER peptide, or analogue thereof, is comprised in a physiological acceptable solution. Suitable physiological acceptable solutions may comprise physiological salt solutions (e.g., 0.9% NaCl) or any other suitable solution for injection and/or infusion. Such physiological solutions may comprise further compounds (e.g., glucose etc.) that may further benefit the human subject, and may also include other pharmaceutical compounds (e.g., vasopressors).

Preferably, the Q-ER peptide is administered at a rate that is at least 10 mg/kg patient weight per hour (mg/kg/hr). Preferably the administration rate is at least 20 mg, at least 30, at least 40 or, most preferably, at least 50 mg/kg/hr. Preferably, the Q-ER peptide is administered for at least 1 hour, more preferably at least 1.5 hours, most preferably at least 2 hours. Preferably, the administration of the Q-ER peptide is at a rate of at least 20 mg/kg/hr and administered for at least 1 hour, more preferably at least 1.5 hours, most preferably at least 2 hours, such as at least 2.5 hours. Preferably, the administration is during surgery. More preferably, the administration is during the entire duration of surgery.

In another embodiment, an Q-ER peptide, or a functional analogue thereof, is provided for any use in accordance with the disclosure as described above, wherein the human subject is admitted to intensive care, and wherein the use improves parameters measured of the human subject, the parameters of the human subject determined to assess to remain in intensive care or not. As shown above, parameters that are assessed when a human patient is in intensive care include parameters related to kidney function and fluid retention, allowing for improved hemodynamic stability. In any case, the use of the Q-ER peptide, or analogue thereof, is to improve such parameters to thereby reduce the length of stay in the intensive care unit. Not only does the use of the Q-ER peptide, or analogue thereof reduce the length of stay in the intensive care, the effect of the use of the Q-ER peptide, or analogue thereof, also reduces the length of stay in the hospital and reduces readmittance into the hospital.

### Further Embodiments

Further embodiment 1: A Q-ER peptide, or a functional analogue thereof, for use in the treatment of a human subject, the use comprising modifying beta-cell function in the human subject.

Further embodiment 2: A Q-ER peptide, or a functional analogue thereof, for use in the treatment of a human subject considered at risk or suffering from beta-cell failure, the use comprising modifying pre-pro-insulin levels in the human subject.

Further embodiment 3: A Q-ER peptide, or a functional analogue thereof, for use in the treatment of a human subject having impaired kidney function, the use comprising modifying fluid retention in the human subject.

Further embodiment 4: A Q-ER peptide, or a functional analogue thereof, for use in the treatment of a human subject, the use comprising modifying inflammation in the human subject.

Further embodiment 5: A Q-ER peptide, or a functional analogue thereof, for use in the treatment of a human subject, the use comprising modifying fluid retention in the human subject.

Further embodiment 6: A Q-ER peptide, or a functional analogue thereof, for use in the treatment of a human subject considered at risk or suffering from excess vasopressor/inotropic use, the use comprising modifying fluid retention in the human subject.

Further embodiment 7: A Q-ER peptide, or a functional analogue thereof, for use in the treatment of a human subject, wherein the human subject is subjected to induced trauma and wherein the use comprises modifying fluid retention in the human subject.

Further embodiment 8: A Q-ER peptide, or a functional analogue thereof, for use in the treatment of a human subject considered at risk or suffering from fluid overload, the use comprising modifying fluid retention in the human subject.

Further embodiment 9: A Q-ER peptide, or a functional analogue thereof, for use in the treatment of a human subject having impaired kidney function, the use comprising modifying fluid retention in the human subject.

Further embodiment 10: A Q-ER peptide, or a functional analogue thereof, for use as in accordance with any one of further embodiments 1-9, wherein the use reduces fluid retention in the human subject.

Further embodiment 11: A Q-ER peptide, or a functional analogue thereof, for use in accordance with any one of further embodiments 1-10 wherein the use comprises a reduced use of vasopressive agents.

Further embodiment 12: A Q-ER peptide, or a functional analogue thereof, for use in accordance with any one of further embodiments 1-11 wherein the use comprises a reduced fluid intake.

Further embodiment 13: A Q-ER peptide, or a functional analogue thereof, for use in accordance with further embodiment 7, wherein the reduced use of vasopressive agents comprises a reduced duration of vasopressive agent use.

Further embodiment 14: A Q-ER peptide, or a functional analogue thereof, for use in accordance with any one of further embodiments 6-9, wherein the subject is subjected to induced trauma.

Further embodiment 15: A Q-ER peptide, or a functional analogue thereof, for use in accordance with any one of further embodiments 8-12 wherein the use improves kidney function in the human subject.

Further embodiment 16: A Q-ER peptide, or a functional analogue thereof, for use in accordance with further embodiment 11, wherein the improved kidney function involves an improved GFR rate.

Further embodiment 17: An AQVG peptide, or a functional analogue thereof, for use in accordance with any one of Further embodiments 6-12, wherein the human subject has impaired kidney function the impaired kidney function being AKI.

Further embodiment 18: A Q-ER peptide, or a functional analogue thereof, for use as in accordance with any one of further embodiments 1-13, wherein the use reduces leakage of plasma and extravasation of blood from the blood to peripheral tissue and/or organs.

Further embodiment 19: A Q-ER peptide, or a functional analogue thereof, for use as in accordance with any one of further embodiments 1-13, wherein the use reduces leakage of plasma from the blood to peripheral tissue and/or organs.

Further embodiment 20: A Q-ER peptide, or a functional analogue thereof, for use as in accordance with any one of further embodiments 1-13, wherein the use reduces extravasation of blood from the blood to peripheral tissue and/or organs.

Further embodiment 21: A Q-ER peptide, or a functional analogue thereof, for use in accordance with any one of further embodiments 1-20, wherein the use is in a human subject suffering from or at risk of heart failure.

Further embodiment 22: A Q-ER peptide, or a functional analogue thereof, for use in accordance with any one of further embodiments 1-21, wherein the use is in a human subject at risk of having edema.

Further embodiment 23: A Q-ER peptide, or a functional analogue thereof, for use in accordance with any one of further embodiments 7 and 14, wherein the human subject has been subjected to induced trauma, the induced trauma being surgery.

Further embodiment 24: A Q-ER peptide, or a functional analogue thereof, for use in accordance with Further embodiment 23, wherein the surgery requires a cardiopulmonary bypass.

Further embodiment 25: A Q-ER peptide, or a functional analogue thereof, for use in accordance with any one of Further embodiments 1-24, wherein the peptide is administered into the bloodstream.

Further embodiment 26: A Q-ER peptide, or a functional analogue thereof, for use in accordance with Further embodiment 25, wherein the peptide is administered at a rate of at least 10 mg/ kg body weight / hour.

Further embodiment 27: A Q-ER peptide, a functional analogue thereof, for use in accordance with further embodiment 25 or further embodiment 26, wherein the peptide is administered for at least 1 hour.

Further embodiment 28: A Q-ER peptide, or a functional analogue thereof, for use in accordance with any one of further embodiments 1-27, wherein the human subject is admitted into intensive care, and wherein the use improves parameters measured of the human subject, the parameters of the human subject determined to assess remaining in intensive care.

Further embodiment 29: A Q-ER peptide, or a functional analogue thereof, for use in accordance with further embodiment 27, wherein the improvement in parameters results in a reduced length of stay at intensive care.

Further embodiment 30: A Q-ER peptide, or a functional analogue thereof, for use as in accordance with any one of further embodiments 1-29, wherein the uses induces vasoconstriction.

Further embodiment 31: A method of treatment comprising administering an Q-ER peptide, or a functional analogue thereof, to a human subject, the human subject being in need of maintaining hemodynamic stability.

Further embodiment 32: A method of treatment comprising administering an Q-ER peptide, or a functional analogue thereof, to a human subject, the human subject being in need of improving hemodynamic stability.

Further embodiment 33: A method of treatment comprising administering an Q-ER peptide, or a functional analogue thereof, to a human subject, the human subject having impaired kidney function, wherein the treatment of administering an Q-ER peptide comprises maintaining or improving hemodynamic stability in the human subject.

Further embodiment 34: A Q-ER peptide, comprising a synthetic peptide or functional analogue thereof, provided with a glutamine (Q) and an elastin-receptor (ER) binding amino acid sequence motif and also comprising at least 50% amino acids selected from the group of autophagy inhibiting amino acids alanine (in one letter code: A), glutamine (Q), glycine (G), valine (V), leucine (L), proline (P), and arginine (R).

Further embodiment 35: A Q-ER peptide, or functional analogue thereof, according to further embodiment 34 comprising at least one amino acid sequence selected from the group of AQ, LQ, GQ, VQ, AQG, LQG, SEQ ID NO:2 (AQGV) and SEQ ID NO: 1 (LQGV).

Further embodiment 36: A Q-ER peptide, consisting of a hepta-, octa-, nona, deca, undeca- or dodeca-peptide according to further embodiment 34 or 35.

Further embodiment 37: A Q-ER peptide according to anyone of further embodiments 34 to 36 provided with at least two glutamines.

Further embodiment 38: A Q-ER peptide according to anyone of further embodiments 34 to 36 selected from the group SEQ ID NO:5 (AQGVAPG), SEQ ID NO:6 (LQGVAPG), SEQ ID NO:7 (AQGVLPG) and SEQ ID NO:8 (LQGVLPG).

Further embodiment 39: A Q-ER peptide according to anyone of further embodiments 34 to 36 selected from the group SEQ ID NO:9 (AQGVAPGQ), SEQ ID NO: 10 (LQGVAPGQ), SEQ ID NO: 11 (AQGVLPGQ) and SEQ ID NO: 12 (LQGVLPGQ).

Further embodiment 40: A Q-ER peptide according to anyone of further embodiments 34 to 36 selected from the group SEQ ID NO:15 (AQGQAPGQ), SEQ ID NO:16 (LQGQAPGQ), SEQ ID NO: 17 (AQGQLPGQ) and SEQ ID NO: 18 (LQGQLPGQ).

Further embodiment 41: A pharmaceutical composition comprising a Q-ER peptide according to any one of further embodiments 34 to 40.

Further embodiment 42: A pharmaceutical composition according to further embodiment 41 additionally comprising an insulin.

Further embodiment 43: A Q-ER peptide according to anyone of further embodiments 3 to 40 or a pharmaceutical composition according to further embodiments 41 or 42 for treatment of impairment of pancreatic beta-cell function.

A suitable receptor for targeting the Q-ER peptides as defined herein is the human elastin-binding-protein (EBP), which can be found in the human elastin receptor complex as the alternatively spliced galactosidase derived from beta-galactosidase, encoded by the GLB1 gene (Uniprot identifier P16278). The isoform 1 of the gene product relates to the beta galactosidase (beta-Gal) whereas the isoform 2 relates to the alternatively spliced galactosidase (EBP or S-Gal). Beta-Gal (isoform 1) cleaves beta-linked terminal galactosyl residues from gangliosides, glycoproteins, and glycosaminoglycans, and is located mainly in the lysosomes.

Isoform 2 (EBP or S-Gal) has little or no beta-galactosidase catalytic activity, but plays functional roles in the formation of extracellular elastic fibers (elastogenesis) and in the development of connective tissue. S-Gal is considered identical to the elastin binding protein (EBP) within the elastin receptor complex (ERC), a major component of the non-integrin cell surface receptor expressed on fibroblasts, smooth muscle cells, chondroblasts, leukocytes, and certain cancer cell types. In elastin producing cells, ERC associates with tropoelastin intracellularly and functions as a recycling molecular chaperone that facilitates the secretions of tropoelastin and its assembly into elastic fibers.

The elastin receptor comprises an alternatively spliced variant of human beta-galactosidase, identified as the elastin binding protein (EBP) or S-Gal. In humans it binds to a hexapeptide x—Gly—x—x—Pro—Gly (xGxxPG) motif in (proteolytic fragments of) extracellular matrix proteins such as elastin and fibrillin-1. The best-known representative of the motif is hexapeptide SEQ ID NO:19 (VGVAPG) (Blanchevoye, C et al. (2013) J Biol Chem 288:1317-28), found in (tropo)elastin, but many other biologically active peptides conforming to the signature sequence xGxxPG, or in some instances xxGxPG, generally called elastin peptides, are reported as agonist. A minimally essential sequence for functionally equivalent biological activity is GxxP, with the peptide at P capable of adopting a type VIII beta-turn. Lactose, and V14 peptide (SEQ ID NO:23 (VVGSPSAQDEASPL) ) corresponding to the binding site of the receptor, are used to antagonise elastin peptide binding. The elastin receptor forms a complex with the elastin binding protein (EBP), neuraminidase (Neu-1) and protective protein-cathepsin A (PPCA) on the cell surface. After binding to its ligand, the complex internalizes to endosomal compartments in the cell and triggers numerous cellular responses. In mice, elastin peptides potentiate atherosclerosis through Neu-1 and regulate IR due to an interaction between Neu-1 and the insulin receptor. Moreover, in mice, PPCA is required for assembly of elastic fibres and inactivation of endothelin-1, impaired activation of endothelin-1 resulting in hypertension.

Recent studies have shown that the Neu-1 component of the ERC complex is responsible for triggering cellular activation. ERC is present on many cell types, including various types of leukocytes, and mesenchymal cells such as vascular smooth muscle cells and fibroblasts. Whereas the hexapeptide SEQ ID NO:19 (VGVAPG), a commonly repeated sequence in human elastin, is the most well-recognized ligand for this receptor, C-peptide, galectin-3 and the beta-2 loop of human choriogonadotropin (hCG) are now herein also recognized as also capable of binding to the ERC. In addition to SEQ ID NO:19 (VGVAPG), peptides that follow the motif GxxPG (where x generally is a hydrophobic amino acid) display chemotaxis for monocytes in vitro (Bisaccia F, et al., Int. J. Pept. Protein Res. 1994;44:332-341, Castiglione Morelli MA, et al., J. Pept. Res. 1997;49:492-499).

Other useful agonists may, for example, be found in silico employing the homology model of the elastin-binding site of human ERC. Blanchevoy et al recently build a homology model of this protein and showed docking of SEQ ID NO:19 (VGVAPG) in this model (Blanchevoye et al, doi: 10.1074/jbc.M112.419929 jbc.M112.419929. ; the contents of which , such as the relevant atomic coordinates of the binding site, are herein included by reference.

In describing protein or peptide composition, structure and function herein, reference is made to amino acids. In the present specification, amino acid residues are expressed by using the following abbreviations. Also, unless explicitly otherwise indicated, the amino acid sequences of peptides and proteins are identified from N-terminal to C-terminal, left terminal to right terminal, the N-terminal being identified as a first residue. Ala: alanine residue; Asp: aspartate residue; Glu: glutamate residue; Phe: phenylalanine residue; Gly: glycine residue; His: histidine residue; Ile: isoleucine residue; Lys: lysine residue; Leu: leucine residue; Met: methionine residue; Asn: asparagine residue; Pro: proline residue; Gln: glutamine residue; Arg: arginine residue; Ser: serine residue; Thr: threonine residue; Val: valine residue; Trp: tryptophane residue; Tyr: tyrosine residue; Cys: cysteine residue. The amino acids may also be referred to by their conventional one-letter code abbreviations; A=Ala; T=Thr; V=Val; C=Cys; L=Leu; Y=Tyr; I=Ile; N=Asn; P=Pro; Q=Gln; F=Phe; D=Asp; W=Trp; E=Glu; M=Met; K=Lys; G=Gly; R=Arg; S=Ser; and H=His.

Peptide shall mean herein a natural biological or artificially manufactured (synthetic) short chain of amino acid monomers linked by peptide (amide) bonds. Glutamine peptide shall mean herein a natural biological or artificially manufactured (synthetic) short chain of amino acid monomers linked by peptide (amide) bonds wherein one of the amino acid monomers is a glutamine.

Chemically synthesized peptides generally have free N- and C-termini. N-terminal acetylation and C-terminal amidation reduce the overall charge of a peptide; therefore, its overall solubility might decrease. However, the stability of the peptide could also be increased because the terminal acetylation/amidation generates a closer mimic of the native protein. These modifications might increase the biological activity of a peptide and are herein also provided. Synthetic GP-domain or xGxxPG-type peptides are often synthesized per classical solid phase synthesis.

### Peptide Synthesis

Synthetic PG-domain or GxxP-type peptides or retro-inverso variants thereof are synthesized according to classical solid phase synthesis. Purity of the peptides is confirmed by high performance liquid chromatography and by fast atom bombardment mass spectrometry. Traditionally, peptides are defined as molecules that consist of between 2 and 50 amino acids, whereas proteins are made up of 50 or more amino acids. In addition, peptides tend to be less well defined in structure than proteins, which can adopt complex conformations known as secondary, tertiary, and quaternary structures. Functional distinctions may also be made between peptides and proteins. In fact, most researchers, as well as this application, use the term peptide to refer specifically to peptides, or otherwise relatively short amino acid chains of up to 50 amino acids (also called oligopeptides), with the term polypeptide being used to describe proteins, or chains of > 50 or much more amino acids.

### Determination of Chemotactic Activity

Human U937 monocytic cells are purchased from the American Type Culture Collection (ATCC catalog number CRL-1593.2, Manassas, Va). Cells are maintained in suspension culture in T-75 flasks containing RPMI 1640 medium supplemented with 10% fetal calf serum and antibiotics, and cultures are split every 3 to 5 days. Three days before use in chemotaxis assays, U937 cells are stimulated to differentiate along the macrophage lineage by exposure to 1 mmol/L dibutyryl cyclic adenosine monophosphate (dbcAMP; Sigma Chemical Co), as described. Cells are washed three times to remove culture medium and then resuspended in chemotaxis medium (Dulbecco’s modified essential medium supplemented with 1% lactalbumin hydrolysate) for plating into assay chambers at a final concentration of 2.5 × 106 cells/mL. Chemotaxis assays are performed in 48-well microchemotaxis chambers (Neuro Probe, Cabin John, Md). The bottom wells of the chamber are filled with 25 mL of the chemotactic stimulus (or medium alone) in triplicate. An uncoated 10-mm-thick polyvinylpyrrolidone-free polycarbonate filter with a pore size of 5 mm is placed over the samples (Neuro Probe). The silicon gasket and the upper pieces of the chamber are applied, and 50 mL of the monocyte cell suspension are placed into the upper wells. Chambers are incubated in a humidified 5% CO2 atmosphere for 3 hours at 37° C., and non-migrated cells are gently wiped away from the upper surface of the filter. The filter is immersed for 30 seconds in a methanol-based fixative and stained with a modified Wright-Giemsa technique (Protocol Hema 3 stain set; Biochemical Sciences, Inc, Swedesboro, NJ) and then mounted on a glass slide. Cells that are completely migrated through the filter are counted under light microscopy, with 3 random high-power fields (HPF; original magnification × 400) counted per well.

Human monocytes are isolated from freshly drawn blood of healthy volunteers using serial Ficoll/Pelastin receptor complex (ERC)oll gradient centrifugation, as described elsewhere. Cells are cultured for 16 hours in RPMI-1640 media supplemented with 0.5% human serum to become quiescent after isolation. Purity of the cells is >95% as determined by flow cytometry analysis. Monocyte chemotaxis is assayed in a 48-well microchemotaxis chamber (Neuroprobe, Gaithersburg, MD) in serum-free media. Wells in the upper and lower chamber are separated by a polyvinylpyrrolidone-free polycarbonate membrane (pore size 5 µm; Costar). Freshly isolated monocytes at a density of 5 × 105/mL are incubated for 2.5 hours with recombinant C-peptide (Sigma), before migrated cells on the bottom face of the filter are stained and counted under the light microscope. Maximal chemotactic activity is measured with 0.1 mmol/L N -formyl-methionyl-leucyl-phenylalanine (f-MLF; Sigma Chemical Co), and checkerboard analysis is used to distinguish chemotaxis from chemokinesis.

Chemotaxis is tested with SEQ ID NO:12 (LQGVLPGQ), SEQ ID NO:11 (AQGVLPGQ), SEQ ID NO:24 (LQGVQPGQ) or SEQ ID NO:25 (AQGVQPGQ). Exposure of cells to lactose is used to specifically dissociate the 67-kD ERC. Controls for lactose included glucose, fructose, and mannose, none of which affect the 67-kD ERC. In each case, monocytes cells are exposed to the relevant concentrations of SEQ ID NO:12 (LQGVLPGQ), SEQ ID NO:11 (AQGVLPGQ), SEQ ID NO:24 (LQGVQPGQ) or SEQ ID NO:25 (AQGVQPGQ) (10-9 to 10-5 mol/L), for 30 minutes before the chemotaxis assays are started. SEQ ID NO:12 (LQGVLPGQ), SEQ ID NO:11 (AQGVLPGQ), SEQ ID NO:24 (LQGVQPGQ) or SEQ ID NO:25 (AQGVQPGQ) induce chemotaxis in a concentration-dependent manner with good activity at 0.1 mmol/L, and it is considerably more potent than the same concentration of f-MLF. Monocytes in the upper wells of the assay chamber are exposed to varying concentrations of SEQ ID NO:12 (LQGVLPGQ), SEQ ID NO:11 (AQGVLPGQ), SEQ ID NO:24 (LQGVQPGQ) or SEQ ID NO:25 (AQGVQPGQ) for 30 minutes before stimulation by SEQ ID NO:19 (VGVAPG) (0.1 nmol/L - 10 nmol/L) in the lower wells. Preincubation of monocytic cells with SEQ ID NO:12 (LQGVLPGQ), SEQ ID NO:11 (AQGVLPGQ), SEQ ID NO:24 (LQGVQPGQ) or SEQ ID NO:25 (AQGVQPGQ) does not alter the chemotactic response to f-MLF. SEQ ID NO:19 (VGVAPG) stimulates a concentration-dependent increase in monocyte migration. SEQ ID NO:12 (LQGVLPGQ), SEQ ID NO:11 (AQGVLPGQ), SEQ ID NO:24 (LQGVQPGQ), or SEQ ID NO:25 (AQGVQPGQ) derived chemotactic activity is eliminated by competition with Val-Gly-Val-Arg-Pro-Gly (SEQ ID NO:19 (VGVAPG)), a repetitive peptide found in human elastin that binds to cellular elastin receptors. Monocyte chemotaxis in response to both SEQ ID NO:19 (VGVAPG) or SEQ ID NO:12 (LQGVLPGQ), SEQ ID NO:11 (AQGVLPGQ), SEQ ID NO:24 (LQGVQPGQ), or SEQ ID NO:25 (AQGVQPGQ) is abolished in the presence of lactose, a galactosugar that specifically dissociates the 67-kD ERC, but it is unaffected by glucose, fructose, or mannose.

Chemotaxis is also assayed by a double micropore membrane system in modified Boyden chambers. The lower compartment containing 180 µl of peptide or fragments thereof at various concentrations is separated from the upper compartment containing 200 µl of cell suspension (5 × 104 cells, such as endothelial cells or smooth muscle cells or pericytes or keratinocytes of fibroblasts or leukocytes per ml medium) by a 10 µm polycarbonate membrane (Millipore, Bedford, MA). The membranes are presoaked in bovine type I collagen (25 micro-g phosphate-buffered saline per ml) (Chemicon International, Temecula, CA) for 24 h at room temperature to facilitate the attachment of cells. The chambers are incubated for 18 h at 37° C. in 5% CO2-balanced air. The chambers are then disassembled and the membrane pairs are stained with hematoxylin. The cell number of a number, such as five, random and non-overlapping fields under a microscope is counted. For all experiments, medium alone in the bottom chamber may serve as the baseline control. To confirm directed cell migration, the concentration gradient between the upper and lower compartments may be abolished by adding various doses of elastin to the cell suspension. Chemotaxis is assayed as described above. Chemotaxis may also be studied in an ex vivo aortic ring assay measuring endothelial cell migration and proliferation

Recombinant elastin binding protein is produced as follows. It is known that the non-sequential alternative splicing of the primary transcript of the β-galactosidase gene generates two mRNAs, one encoding the precursor of the lysosomal enzyme (β-gal) and the second encoding an enzymatically inactive protein (S-gal or elastin binding protein), which is not targeted to the lysosomes. In the S-gal-encoding mRNA, exons 3, 4, and 6 are spliced out, and exon 5 is shifted in frame, thus creating a unique region encoding a 32-amino acid sequence in S-gal, which differs from its counterpart encoded by exon 5 of active β-gal, and contains an elastin-peptide binding domain. S-gal cDNA clone is constructed with routine procedures. To construct the full-length alternatively spliced cDNA clone (1986 bp) reflecting the sequence described by Morreau and colleagues, poly(A)+ mRNA is isolated from cultured normal human skin fibroblasts using a Quick Prep mRNA purification kit from Pharmacia. This mRNA (500 ng) is reverse-transcribed using random hexamers and superscript reverse transcriptase (Life Technologies, Inc.). To isolate overlapping fragments of the cDNA, two polymerase chain reactions (PCR) are carried out. The 5′ portion of the cDNA (357 bp) is amplified using the primers 5′- SEQ ID NO:54 (GGTGGTCATGCCGGGGTTCCT) -3′ and 5′- SEQ ID NO:55 (ATGTTGCTGCCTGCACTGTT) -3′. The primers 5′- SEQ ID NO:56 (CCATCCAGACATTACCTGGC) -3′ and 5′- SEQ ID NO:57 (CCCTCACACATTCCAGGTGGT) -3′ are used to amplify the 3′ fragment of the cDNA (1598 bp). The reactions are carried out on a Perkin-Elmer thermal cycler using an annealing temperature of 55° C. The fragments are gel-purified and ligated into the EcoRV site of pBluescript SK+. The respective fragments contain a 115-bp overlapping sequence at their respective 3′ and 5′ ends. In addition, the absence of the initial 27 bp located at the 5′ end of the 5′ fragment and 119 bp at the 3′ end of the 3′ fragment is detected (attributed to primer positioning in the initial PCR reaction). Final assembly of the full-length clone eliminates the overlapping segment by employing a common PvuII site found in the overlapping region between the two portions of S-gal. Complete double digestion of the 5′ clone with the restriction enzymes KpnI and PvuII yields a 316-bp fragment representing the 5′ segment (i.e., 5′ to the PvuII site) of S-gal, which includes an additional 57 bp of vector at its 5′ end. The KpnI digestion creats a 3′ overhang, which is blunt-ended by using Pfu DNA polymerase. The 316-bp 5′ fragment and a PvuII-digested 3′ clone are then both agarose gel-purified, gene-cleaned, and ligated. The ligation products are transformed into bacterial cell and grown on LB-AMP plates. Restriction digests with XhoI andPvuII confirm the correct orientation of the short 5′ segment of S-gal in the new construct. In vitro transcription/translation is done in accordance to the protocols provided by Promega. S-gal cDNA (5 µg) in pGEM-3Z is linearized (digested with XbaI), and in vitro transcription is conducted. This is followed by in vitro translation using 2 µl of RNA substrate in a nuclease-treated rabbit reticulocyte lysate (minus microsomal membranes and protease inhibitors) in the presence of 0.8 mCi/ml [35S]methionine ([35S]Met). The translation mix (minus mRNA) is used as control. The supernatants are directly analyzed by SDS-polyacrylamide gel electrophoresis (SDS-PAGE), followed by autoradiography to detect for the presence of [35S]Met-labeled reaction products and to compare molecular size. The reaction products are further characterized using immunoprecipitation with antibodies recognizing β-gal, S-gal, and elastin binding protein, and then by elastin affinity columns.

Useful synthetic Q-ER peptides herein are listed with their sequence identifiers below in table 1. In particular, a peptide of Table 1 having at least one human elastin receptor binding motif GxxPG, and having at least one amino acid Q, is herein provided for use in treatment of a human suspected of having or having an acute inflammatory condition or for use in treatment of a human suspected of having or having a microvascular complication, such as in impaired beta-cell function seen with diabetes type 1 or end-phase type 2, and in hemodynamic conditions such as impaired kidney function, fluid retention, and/or extravasation of leucocytes and blood plasma.

TABLE 1

SEQ ID NO:1 (LQGV)
SEQ ID NO:2 (AQGV)
SEQ ID NO:3 (QGVLPG)
SEQ ID NO:4 (QGVAPG)
SEQ ID NO:5 (AQGVAPG)
SEQ ID NO:6 (LQGVAPG)
SEQ ID NO:7 (AQGVLPG)
SEQ ID NO:8 (LQGVLPG)
SEQ ID NO:9 (AQGVAPGQ)
SEQ ID NO:10 (LQGVAPGQ)
SEQ ID NO:11 (AQGVLPGQ)
SEQ ID NO:12 (LQGVLPGQ)
SEQ ID NO:13 (LQGQAPG)
SEQ ID NO:14 (QGQAPG)
SEQ ID NO:15 (AQGQAPGQ)
SEQ ID NO:16 (LQGQAPGQ)
SEQ ID NO: 17 (AQGQLPGQ)
SEQ ID NO:18 (LQGQLPGQ)
SEQ ID NO:19 (VGVAPG)
SEQ ID NO:20 (LQVGLLPG)
SEQ ID NO:21 (AQPGQLPG)
SEQ ID NO:22 (AQGVQPG)
SEQ ID NO:23 (VVGSPSAQDEASPL)
SEQ ID NO:24 (LQGVQPGQ)
SEQ ID NO:25 (AQGVQPGQ)
SEQ ID NO:26 (VQGVLPG)
SEQ ID NO:27 (VQGVAPG)
SEQ ID NO:28 (GQGVLPG)
SEQ ID NO:29 (GQGVAPG)
SEQ ID NO:30 (LQGQLPG)
SEQ ID NO:31 (VQGQLPG)
SEQ ID NO:32 (VQGVLPGQ)
SEQ ID NO:33 (VQGVAPGQ)
SEQ ID NO:34 (AQGQLPG)
SEQ ID NO:35 (GQGQLPG)
SEQ ID NO:36 (GQGVLPGQ)
SEQ ID NO:37 (GQGVAPGQ)
SEQ ID NO:38 (VQGQLPGQ)
SEQ ID NO:39 (GQGQLPGQ)
SEQ ID NO:40 (LQGLQPGA)
SEQ ID NO:41 (LQGAQPGQ)
SEQ ID NO:42 (LQGLQPGQ)
SEQ ID NO:43 (VQGLQPGQ)
SEQ ID NO:44 (VQGVQPGQ)
SEQ ID NO:45 (VQGAQPGQ)
SEQ ID NO:46 (VQGGQPGQ)
SEQ ID NO:47 (AQGLQPGQ)
SEQ ID NO:48 (AQGAQPGQ)
SEQ ID NO:49 (AQGGQPGQ)
SEQ ID NO:50 (GQGLQPGQ)
SEQ ID NO:51 (GQGVQPGQ)
SEQ ID NO:52 (GQGAQPGQ)
SEQ ID NO:53 (GQGGQPGQ)

Useful nucleic acids as synthesised herein in Table 2.

TABLE 2

5′- SEQ ID NO:54 (GGTGGTCATGCCGGGGTTCCT) -3′
5′- SEQ ID NO:55 (ATGTTGCTGCCTGCACTGTT) -3′
5′- SEQ ID NO:56 (CCATCCAGACATTACCTGGC) -3′
5′- SEQ ID NO:57 (CCCTCACACATTCCAGGTGGT) -3′

TABLE 3

Preferred Q-ER peptides having a GxxPG elastin-receptor-binding motif and conforming amino acids GQ at position xx.
SEQ ID NO:46 (VQGGQPGQ)
SEQ ID NO:49 (AQGGQPGQ)
SEQ ID NO:53 (GQGGQPGQ)

TABLE 4

Preferred Q-ER peptides having a GxxPG elastin-receptor-binding motif and conforming amino acids VQ at position xx.
SEQ ID NO:51 (GQGVQPGQ)
SEQ ID NO:44 (VQGVQPGQ)
SEQ ID NO:24 (LQGVQPGQ)
SEQ ID NO:22 (AQGVQPG)
SEQ ID NO:25 (AQGVQPGQ)

TABLE 5

Preferred Q-ER peptides having a GxxPG elastin-receptor-binding motif and conforming amino acids VA at position xx.
SEQ ID NO:4 (QGVAPG)
SEQ ID NO:5 (AQGVAPG)
SEQ ID NO:6 (LQGVAPG)
SEQ ID NO:9 (AQGVAPGQ)
SEQ ID NO:10 (LQGVAPGQ)
SEQ ID NO:27 (VQGVAPG)
SEQ ID NO:29 (GQGVAPG)
SEQ ID NO:33 (VQGVAPGQ)
SEQ ID NO:37 (GQGVAPGQ)

TABLE 6

Preferred Q-ER peptides having a GxxPG elastin-receptor-binding motif and conforming amino acids VL at position xx. ]
SEQ ID NO:3 (QGVLPG)
SEQ ID NO:7 (AQGVLPG)
SEQ ID NO:8 (LQGVLPG)
SEQ ID NO:11 (AQGVLPGQ)
SEQ ID NO:12 (LQGVLPGQ)
SEQ ID NO:26 (VQGVLPG)
SEQ ID NO:28 (GQGVLPG)
SEQ ID NO:32 (VQGVLPGQ)
SEQ ID NO:36 (GQGVLPGQ)

TABLE 7

Preferred Q-ER peptides having a GxxPG elastin-receptor-binding motif and conforming amino acids QA at position xx. ]
SEQ ID NO:13 (LQGQAPG)
SEQ ID NO:14 (QGQAPG)
SEQ ID NO:15 (AQGQAPGQ)
SEQ ID NO:16 (LQGQAPGQ)

TABLE 8

Preferred Q-ER peptides having a GxxPG elastin-receptor-binding motif and conforming amino acids AQ at position xx.
SEQ ID NO:45 (VQGAQPGQ)
SEQ ID NO:41 (LQGAQPGQ)
SEQ ID NO:48 (AQGAQPGQ)
SEQ ID NO:52 (GQGAQPGQ)

TABLE 9

Preferred Q-ER peptides having a GxxPG elastin-receptor-binding motif and conforming amino acids QL at position xx.
SEQ ID NO: 17 (AQGQLPGQ)
SEQ ID NO:18 (LQGQLPGQ)
SEQ ID NO:21 (AQPGQLPG)
SEQ ID NO:30 (LQGQLPG)
SEQ ID NO:31 (VQGQLPG)
SEQ ID NO:34 (AQGQLPG)
SEQ ID NO:35 (GQGQLPG)
SEQ ID NO:38 (VQGQLPGQ)
SEQ ID NO:39 (GQGQLPGQ)

TABLE 10

Preferred Q-ER peptides having a GxxPG elastin-receptor-binding motif and conforming amino acids LQ at position xx.
SEQ ID NO:20 (LQVGLQPG)
SEQ ID NO:40 (LQGLQPGA)
SEQ ID NO:42 (LQGLQPGQ)
SEQ ID NO:43 (VQGLQPGQ)
SEQ ID NO:47 (AQGLQPGQ)
SEQ ID NO:50 (GQGLQPGQ)

TABLE 11

Preferred Q-ER peptides having a GxxPG elastin-receptor-binding motif and furthermore provided with a peptide sequence with SEQ ID NO: 1 (LQGV).
SEQ ID NO:6 (LQGVAPG)
SEQ ID NO:8 (LQGVLPG)
SEQ ID NO:10 (LQGVAPGQ)
SEQ ID NO:12 (LQGVLPGQ)
SEQ ID NO:24 (LQGVQPGQ)

TABLE 12

Preferred Q-ER peptides having a GxxPG elastin-receptor-binding motif and furthermore provided with a peptide sequence with SEQ ID NO:2 (AQGV).
SEQ ID NO:5 (AQGVAPG)
SEQ ID NO:7 (AQGVLPG)
SEQ ID NO:9 (AQGVAPGQ)
SEQ ID NO:11 (AQGVLPGQ)
SEQ ID NO:22 (AQGVQPG)
SEQ ID NO:25 (AQGVQPGQ)

The disclosure also provides synthetic peptides wherein anyone peptide from Table 1 list of Q-ER peptides has been repeated at least once, optionally the repeats are separated by a linker, such a linker may comprise one or more amino acids, such as one or more amino acids selected from the group of glycine, alanine, leucine, valine, isoleucine or glutamine.

The disclosure also provides synthetic peptides wherein at two or three peptides with the pentapeptide motif GxxPG found in above listed peptides in Table 1 are included at least once, optionally the peptides with PG-domain repeats are separated by a linker, such a linker may comprise one or more amino acids, such as one or more amino acids selected from the group of glycine, alanine, leucine, valine, isoleucine or glutamine.

### Pharmaceutical Q-ER Compositions

### Example 1

Peptide SEQ ID NO: 11 (AQGVLPGQ)
To prepare 10 ml of the composition, mix
Peptide SEQ ID NO: 11 (AQGVLPGQ)--500 mg
M-Kreosol--25 mg
Glycerol--160 mg
Water and either 10% hydrochloric acid or 10% sodium hydroxide sufficient to make a composition volume of 10 ml and a final pH of 7.0-7.8. Optionally, an acid resistant capsule is filled with above composition.

### Example 2

Peptide SEQ ID NO: 12 (LQGVLPGQ)
To prepare 10 ml of the composition, mix
Peptide SEQ ID NO: 12 (LQGVLPGQ)--500 mg
M-Kreosol--25 mg
Glycerol--160 mg
Water and either 10% hydrochloric acid or 10% sodium hydroxide sufficient to make a composition volume of 10 ml and a final pH of 7.0-7.8 Optionally, an acid resistant capsule is filled with above composition.

### Example 3

Peptide SEQ ID NO:47 (AQGLQPGQ)
To prepare 10 ml of the composition, mix
Peptide SEQ ID NO:47 (AQGLQPGQ)--500 mg
M-Kreosol--25 mg
Glycerol--160 mg
Water and either 10% hydrochloric acid or 10% sodium hydroxide sufficient to make a composition volume of 10 ml and a final pH of 7.0-7.8. Optionally, an acid resistant capsule is filled with above composition.

### Example 4

Peptide SEQ ID NO:40 (LQGLQPGQ)
To prepare 10 ml of the composition, mix
Peptide SEQ ID NO:40 (LQGLQPGQ)--500 mg
M-Kreosol--25 mg
Glycerol--160 mg
Water and either 10% hydrochloric acid or 10% sodium hydroxide sufficient to make a composition volume of 10 ml and a final pH of 7.0-7.8. Optionally, an acid resistant capsule is filled with above composition.

### Example 5

Peptide SEQ ID NO: 11 (AQGVLPGQ) and insulin
To prepare 10 ml of the composition, mix
Human Insulin (28 U/mg)--1000 U
Peptide SEQ ID NO:11 (AQGVLPGQ)--500 mg
M-Kreosol--25 mg
Glycerol--160 mg
Water and either 10% hydrochloric acid or 10% sodium hydroxide sufficient to make a composition volume of 10 ml and a final pH of 7.0-7.8

### Example 6

Peptide SEQ ID NO: 12 (LQGVLPGQ) and insulin
To prepare 10 ml of the composition, mix
Human Insulin (28 U/mg)--1000 U
Peptide SEQ ID NO:12 (LQGVLPGQ)--500 mg
M-Kreosol--25 mg
Glycerol--160 mg
Water and either 10% hydrochloric acid or 10% sodium hydroxide sufficient to make a composition volume of 10 ml and a final pH of 7.0-7.8

### Example 7

Peptide SEQ ID NO:47 (AQGLQPGQ) and insulin
To prepare 10 ml of the composition, mix
Human Insulin (28 U/mg)--1000 U
Peptide SEQ ID NO:47 (AQGLQPGQ)--500 mg
M-Kreosol--25 mg
Glycerol--160 mg
Water and either 10% hydrochloric acid or 10% sodium hydroxide sufficient to make a composition volume of 10 ml and a final pH of 7.0-7.8

### Example 8

Peptide SEQ ID NO:40 (LQGLQPGQ) and insulin
To prepare 10 ml of the composition, mix
Human Insulin (28 U/mg)--1000 U
Peptide SEQ ID NO:40 (LQGLQPGQ)--500 mg
M-Kreosol--25 mg
Glycerol--160 mg
Water and either 10% hydrochloric acid or 10% sodium hydroxide sufficient to make a composition volume of 10 ml and a final pH of 7.0-7.8

### Example 9

Peptide SEQ ID NO:5 (AQGVAPG)
To prepare 10 ml of the composition, mix
Peptide SEQ ID NO:5 (AQGVAPG)—40 mg
PBS or 0.9% NaCl sufficient to make a composition volume of 10 ml.

### Example 10

Peptide SEQ ID NO:6 (LQGVAPG)
To prepare 10 ml of the composition, mix
Peptide SEQ ID NO:6 (LQGVAPG)—40 mg
PBS or 0.9% NaCl sufficient to make a composition volume of 10 ml

### Example 11

Peptide SEQ ID NO:7 (AQGVLPG)
To prepare 10 ml of the composition, mix
Peptide SEQ ID NO:7 (AQGVLPG)—40 mg
PBS or 0.9% NaCl sufficient to make a composition volume of 10 ml

### Example 12

Peptide SEQ ID NO:8 (LQGVLPG)
To prepare 10 ml of the composition, mix
Peptide SEQ ID NO:8 (LQGVLPG)—40 mg
PBS or 0.9% NaCl sufficient to make a composition volume of 10 ml

Example 13 Inhibition of autophagy by selected amino acids.

Autophagy is a degradation pathway that delivers extra cellular and cytoplasmic materials to lysosomes via double-membraned vesicles designated autophagosomes. Cytoplasmic constituents are sequestered into autophagosomes, which subsequently fuse with lysosomes, where the cargo is degraded. Extracellular materials are taken up by endocytosis or phagocytosis, which subsequently fuse with lysosomes, again where the cargo is degraded. Autophagy is a crucial mechanism involved in many aspects of cell function, including cellular metabolism and energy balance; and alterations in autophagy have been linked to various human pathological processes. Autophagy is a natural mechanism in which the cell removes and degrades cellular components with autolysosomes. It is a popular research area because autophagy is related to many physical and pathological processes. In this way, cells have a constant turnover of proteins that recycle most amino acids over time. Homeostasis is achieved through exchange of essential amino acids with non-essential amino acids and the transfer of amino groups from oxidized amino acids to amino acid biosynthesis. This homeostatic condition is maintained through an active mTORC1 complex. Under amino acid depletion, mTORC1 is inactivated.

The disclosure provides that some amino acids control proteogenesis (mTOR kinases) or proteolysis (autophagy) more than others. The mechanistic target of rapamycin complex I (mTORC1) is a central regulator of cellular and organismal growth and this pathway is implicated in the pathogenesis of many human diseases. mTORC1 promotes growth in response to the availability of nutrients, such as amino acids, which drive mTORC1 to the lysosomal surface, its site of activation. How amino acid levels are communicated to mTORC1 is only recently coming to light by the discovery of a lysosome-based signaling system composed of the Rag GTPases and Ragulator, v-ATPase, GATOR and Folliculin complexes. To stimulate cell growth, mTORC1 relies on its downstream effectors to coordinately promote anabolic programs such as mRNA translation (Ma XM, Blenis J. Molecular mechanisms of mTOR-mediated translational control. Nat Rev Mol Cell Biol. 2009; 10:307-318) underlying proteogenesis, cellular differentiation and cell- and cell-organelle growth and repress, lower or inhibit catabolic programs such as autophagy (Rabinowitz JD, White E. Autophagy and metabolism. Science. 2010;330:1344-1348), underlying proteolysis and cell- or cell-organelle decay, thereby avoiding a futile cycle of uncoordinated (anabolic) synthesis and (catabolic) degradation. In general, under low amino acid conditions Ragulator is found in an inhibitory state with the v-ATPase and GATOR1 exerts its GAP activity toward RagA, keeping this GTPase in the inactive GDP-bound state that is not sufficient to recruit mTORC1. Insulin signaling inhibits TSC complex translocation to the lysosomal surface where it functions as a GAP for Rheb, inactivating this G protein. Upon amino acid stimulation, GATOR1 may be inhibited by GATOR2 and Ragulator and v-ATPase undergo a conformational change unleashing the GEF activity of Ragulator toward RagA, while the folliculin complex promotes RagC GTP hydrolysis. The now active heterodimer, consisting of GTP-bound RagA and GDP-loaded RagC, recruits mTORC1 to the lysosomal surface, where it interacts with and is activated by Rheb. mTORC1 is regulated by the small GTPase Rheb, which resides at the lysosomal surface where it functions as a potent stimulator of the mTORC1 kinase activity. In essence, autophagy is an intracellular degradation system, where dysfunctional proteins and organelles are degraded. In this process, aggregated dysfunctional proteins are taken up by cells through endocytosis or phagocytosis, or intercellularly surrounded by the double membrane from lysosomal vesicles to form an autophagosome wherein such degradation is achieved, typically involving hydrolysis. DALGreen is used to detect Q-ER-peptide induced autophagy in live cells. As autophagy is an intracellular degradation system, in this process, aggregated dysfunctional proteins are surrounded by the double membrane to form an autophagosome. Methods to detect and quantify (amino acid mediated) inhibition of autophagy are known in the art (see, for example, Zhang, Ziyan, Rajat Singh, and Michael Aschner. “Methods for the Detection of Autophagy in Mammalian Cells.” Current protocols in toxicology 69.1 (2016): 20.12. 1-20.12. 26, and references below in Table 13). For example, DALGreen, which is a small hydrophobic molecule, passes the plasma membrane of live cells and is incorporated in the autophagosome. After a lysosome fuses with the autophagosome, the environment in the autolysosome become acidic. DALGreen fluoresce stronger as acidity increases. The quality of this dye enables live cell imaging with a fluorescence microscopy and quantitative assay of Q-ER mediated autophagy by flow cytometry. In other autophagy studies with Q-ER peptide, autophagy is determined through, LC3-I and LC3-II detection to track the autophagy process, allowing quantifying Q-ER mediated autophagy with LC3. LC3 is a key protein in the autophagy pathway. Once synthesized, cytoplasmic LC3 is processed by Atg4 at the C-terminal, forming cytoplasmic LC3-I (16 KDa). Upon autophagy signal, LC3-I is conjugated by ubiquitin-like proteins Atg7 and Atg3 to the lipid phosphatidylethanolamine (PE), generating lipidated LC3-II (14 KDa). The lipidated LC3-II binds to both inner and outer membranes of autophagosomes, with the former being degraded after fusion with lysosomes; whereas LC3 on the outer membrane is deconjugated by ATG4 and returns to the cytosol. Thus, LC3-I to LC3-II conversion and lysosomal degradation of LC3-II reflect the progression of autophagy, and immunoblotting is used to monitor changes in LC3 amount after Q-ER targeting to a cell.

Recent and older data (see also Table 13) identify leucine (L), valine (V), isoleucine (I), alanine (A), glutamine (Q), arginine (R), glycine (G), proline (P), either alone or in combination, as more potent activators of mTOR or inhibitors of autophagy than other amino acids, such as glutamate (E), threonine (T), serine (S), lysine (K), threonine (T), phenylalanine (F), tyrosine (Y), and methionine (M) that have been reported to have no or opposite effects. Hence, as herein provided for inclusion in a Q-ER (Q-vh/vhh) peptide according to the disclosure, leucine (L), valine (V), isoleucine (I), alanine (A), glutamine (Q), arginine (R), glycine (G), proline (P), either alone or (preferably) in combination, are most preferred activators of mTOR or inhibitors of autophagy for use in human cells, for packaging and targeting to cells. Amino acids leucine (L), alanine (A), glutamine (Q), and proline (P are reported to have most prominent mTOR associated autophagic inhibitory effects on human cells (AJ Meijer et al Amino Acids 2015, 47, 2037-2063). Glycine (Zhong Z, Wheeler MD, Li X, Froh M, Schemmer P, Yin M, Bunzendaul H, Bradford B, Lemasters JJ. 1-Glycine: a novel antiinflammatory, immunomodulatory, and cytoprotective agent. Curr Opin Clin Nutr Metab Care 6: 229-240, 2003) improves amino-acid-stimulated mammalian target of rapamycin (mTOR) complex 1 activation. Hence, as herein provided for inclusion in a Q-ER (Q-vh/vhh) peptide according to the disclosure, leucine (L), alanine (A), glutamine (Q), glycine (G) and proline (P), either alone or (preferably) in combination, are most preferred activators of mTOR or inhibitors of autophagy for use in human cells.

TABLE 13

| | | | | | |
|---|---|---|---|---|---|
| | | | mTOR kinases | autophagy | |
| L-Glycine | Gly | G | | DOWN | Bluem J Biol Chem 2007 37783 |
| | | | | DOWN | Qin et al http://en.cnki.com.cn/Article en/CJFDTOTAL-NJYK200912002.htm |
| L-Alanine | Ala | A | | DOWN | Proc. Natl. Acad. Sci. USA Vol. 76, No. 7, pp. 3169-3173, July 1979 |
| L-Proline | Pro | P | activate | | Washington Am J Physiol Cell Physiol 2010 298 C982 |
| L-Valine | Val | V | activate | | Maria Dolors Sans et al J. Nutr. 136: 1792-1799, 2006. |
| L-Isoleucine | Ile | I | activate | DOWN | Doi J Nutr 135 2102-8 Biochen Biophys Acta 2008 1115 |
| L-Isoleucine | | | | | Maria Dolors Sans et al J. Nutr. 136: 1792-1799, 2006. |
| L-Leucine | Leu | L | activate | DOWN | Ijichi et al Bioc Biophys Res com 2003 303 59 |
| L-Leucine | | | | | Maria Dolors Sans et al J. Nutr. 136: 1792-1799, 2006. |
| L-Glutamine | Gln | Q | activate | DOWN | Amino Acids 2009 73 111 |
| L-Glutamine | | | activate | DOWN | Kim Biol Reprod 2011 84 1139 |
| L-Arginine | Arg | R | activate | DOWN | Ban et al. Int J Mol Med 2004 13:537-43 |
| L-Arginine | | | activate | DOWN | Kim Biol Reprod 2011 84 79 |
| L-Tyrosine | Tyr | Y | | | |
| L-Lysine | Lys | K | down | | Prizant J Cell Biochem 2008 1 1038 |
| L-Tryptophan | Trp | W | | | |
| L-Cystine | Cys | C | | | |
| L-Serine | Ser | S | | | |
| L-Threonine | Thr | T | down | | Prizant J Cell Biochem 2008 1 1038 |
| L-Asparagine | Asn | N | | | |
| L-Aspartic acid | Asp | D | | | |
| L-Methionine | Met | M | partial inhibit | | Stubs J Endocrinol 2002 174 335 |
| L-Histidine | His | H | down | | Prizant J Cell Biochem 2008 1 1038 |
| L-Phenylalanine | Phe | F | | | |
| L-Glutamic Acid | Glu | E | | | |

### Example 14

Q-ER peptide provided with an elastin-receptor binding motif and enriched with amino acids that inhibit autophagy reduce diabetes type 1 in mice.

The non-obese diabetic (NOD) mouse spontaneously develops type 1 diabetes (T1D) and has served as a model for understanding the genetic and immunological basis, and treatment, of T1D. NOD mice are used in these studies. Most diabetic NOD mice suffer from insulin deficiency diabetes, with the underlying cause of the pancreatic beta-cell loss or destruction most likely a result of an inflammatory process in the micro-vasculature surrounding endocrine beta-cell tissues and genetically accelerated autoimmunity is suspected. Diabetes is assessed by measurement of the venous blood glucose level using an Abbott Medisense Precision glucometer. Mice are considered diabetic after two consecutive glucose measurements ≥11 mmol/l (200 mg/dl). Onset of diabetes is dated from the first consecutive reading of ≥11 mmol/l. In vivo treatment: groups of 14 weeks-old female NOD mice (n=12 per group) are treated with synthetic peptide SEQ ID NO:5 (AQGVAPG), SEQ ID NO:6 (LQGVAPG), SEQ ID NO:7 (AQGVLPG), SEQ ID NO:8 (LQGVLPG) or vehicle (PBS), respectively. Treatment is done by injecting 250 µg peptide diluted in PBS three times per week intraperitoneally (i.p.). Control mice are treated with PBS only. The treatment is discontinued when all PBS treated NOD mice in the experiment have developed diabetes. This is generally at the age of 19 weeks. The peptide and PBS treatments are done for a maximum of five weeks. In instances of hyperglycaemia of ≥20 mmol/l the mice are killed to avoid prolonged discomfort. The remainder mice are kept alive up to the age of 35 weeks without any further treatment. These experiments show that peptide treatment significantly reduces beta-cell failure with diabetes development, as measured by delayed occurrence of diabetes after the age of 17 weeks.

### Example 15

Q-ER peptides provided with an elastin-receptor binding motif and enriched with amino acids that inhibit autophagy reduce SIRS.

Severe hemorrhagic shock followed by resuscitation induces a massive inflammatory response, which may culminate into systemic inflammatory response syndrome, multiple organ dysfunction syndrome and finally death. Treatments that effectively prevent this inflammation are limited so far. In a previous study, it was demonstrated that synthetic oligopeptides LQGV or AQGV related to the primary structure of human chorionic gonadotropin can inhibit the inflammatory response and mortality that follow high-dose lipopolysaccharide induced inflammation. Considering this powerful anti-inflammatory effect, it was investigated whether administration of 0.5 ml i.p. of synthetic heptapeptide SEQ ID NO:5 (AQGVAPG), SEQ ID NO:6 (LQGVAPG), SEQ ID NO:7 (AQGVLPG), SEQ ID NO:8 (LQGVLPG), dissolved in 0.9% NaCl at a concentration of 25 mg/ml, or vehicle (0.9% NaCl), respectively, during the SIRS-phase of hemorrhagic shock, is able to attenuate the inflammatory response associated with this condition.

Hemorrhagic shock is induced in groups of 6 rats for 60 minutes by blood withdrawal until a mean arterial pressure of 40 mmHg is reached. Thirty minutes after induction of hemorrhagic shock, rats received a single injection with one of synthetic heptapeptide SEQ ID NO:5 (AQGVAPG), SEQ ID NO:6 (LQGVAPG), SEQ ID NO:7 (AQGVLPG), SEQ ID NO:8 (LQGVLPG), or 0.9% NaCl solution, as control. Treatment with synthetic heptapeptide SEQ ID NO:5 (AQGVAPG), SEQ ID NO:6 (LQGVAPG), SEQ ID NO:7 (AQGVLPG), SEQ ID NO:8 (LQGVLPG) prevents systemic release of TNF-α and IL-6 and is associated with reduced TNF-α, IL-6 and E-selectin mRNA transcript levels in the liver, with reduced neutrophil infiltration into the liver and is associated with reduced liver damage.

Animals. Adult male specific pathogen-free Wistar rats weighing 350-400 g are used. Rats are housed under barrier conditions at 25° C. with a twelve-hour light/dark cycle, and are allowed food and water ad libitum. The experimental protocol adheres to the rules laid down in national law that serves the implementation of “Guidelines on the protection of experimental animals” by the Council of Europe (1986), Directive 86/609/EC. hCG-related synthetic oligopeptides. Rats are food deprived overnight before the start of the experiment, but are allowed water ad libitum. Rats are anesthetized using a mixture of N2 O/O2 /isoflurane (Pharmachemie B.V., Haarlem, The Netherlands). Body temperature is continuously maintained at 37.5° C. by placing the rats on a thermo controlled “half-pipe” (UNO, Rotterdam, The Netherlands). Endotracheal intubation is performed, and rats are ventilated at 60 breaths per minute with a mixture of N2 O/O2 /isoflurane. Polyethylene tubes (PE-50, Becton Dickinson; St. Michielsgestel, The Netherlands) are flushed with heparin and placed via the right carotid artery in the aorta and in the right internal jugular vein. A 5 cm midline laparotomy is performed and a supra pubic catheter is inserted to monitor urine production. Hemorrhagic shock is induced by blood withdrawal, reducing the circulating blood volume until a mean arterial pressure (MAP) of 40 mmHg is reached. This level of hypotension is maintained for 60 minutes. Thirty minutes after the induction of hemorrhagic shock, rats received a single intravenous (IV) bolus injection of heptapeptide solution or vehicle. Sixty minutes after induction of hemorrhagic shock, rats are resuscitated by four times their shed blood volume over a period of 30 minutes to normalize the MAP, and monitored for another 120 minutes after which they are sacrificed. The rats receive no heparin before or during the experiment. Sham animals are subjected to the same surgical procedure as the hemorrhagic shock animals, but without blood withdrawal and administration of heptapeptide.

Example 16 Q-ER peptides provided with an elastin-receptor binding motif and enriched with amino acids that inhibit autophagy reduce acute inflammation.

Short synthetic Q-ER peptides having been provided with an elastin receptor binding motif (examples of which are shown in tables above) comprising distinct and selected amino acids that preferentially activate mTOR or preferentially inhibit autophagy reduce inflammatory activity of white blood cells and may reduce acute systemic inflammation as a whole. Examples of peptides that are enriched with these above amino acids and down-regulate inflammation disease herein provided. Other peptides are now easily derived, preferably by generating or synthesizing small peptides by combining amino acids that preferentially activate mTOR or preferentially inhibit autophagy, preferably selected from the group of A, G, L, V, Q and P, into strings of Q-ER peptides that also have been provided with a GxxPG motif allowing targeting the elastin-receptor-complex.

Example 17 Q-ER peptides provided with an elastin-receptor binding motif and enriched with amino acids that inhibit autophagy reduce vascular permeability.

Prevention of VEGF-mediated microvascular permeability in mice is, for example, achieved by repeated administration of 0.5 ml (i.p) of heptapeptide with SEQ ID NO:7 (AQGVLPG) dissolved in 0.9% NaCl at a concentration of 25 mg/ml in diabetic mice, at three-weekly dosing for 4 weeks. VEGF-induced vascular leakage is investigated in the skin of diabetic mice using a Miles vascular permeability assay. Such heptapeptide administration is showing the protective role of the short synthetic Q-ER peptide of the disclosure, (various examples of which are shown in tables above) comprising distinct and selected amino acids that preferentially activate mTOR or preferentially inhibit autophagy, against increased microvascular permeability mediated by vascular endothelial growth factor (VEGF)-induced reactive oxygen species (ROS) generation in diabetes.

Example 18 Q-ER peptides provided with an elastin-receptor binding motif and enriched with amino acids that inhibit autophagy reduce leukocyte extravasation.

Short synthetic Q-ER peptides, (examples of which are shown in tables above) comprising distinct and selected amino acids preferentially activate mTOR or preferentially inhibit autophagy reduce leukocyte extravasation. Heptapeptides (SEQ ID NO:6 (LQGVAPG), SEQ ID NO:7 (AQGVLPG), SEQ ID NO:8 (LQGVLPG)) are dissolved in 0.9% NaCl at a concentration of 15 mg/mL. Adult male specific pathogen-free Wistar rats (Harlan CPB, Zeist, The Netherlands) weighing 350 to 400 g are used. Rats are housed under barrier conditions at 25° C. with a 12-h light-dark cycle and are allowed food and water ad libitum. The experimental protocol is approved by the Animal Experiments Committee under the Dutch Experiments on Animals Act and adhered to the rules laid down in this national law that serves the implementation of “Guidelines on the Protection of Experimental Animals” by the Council of Europe (1986; directive 86/609/EC).

Rats are deprived of food overnight before the start of the experiment but are allowed water ad libitum. Rats are anesthetized using a mixture of N2O/O2/isoflurane (Pharmachemie B.V., Haarlem, The Netherlands). Body temperature is continuously maintained at 37.5° C. by placing the rats on a thermo-controlled “half-pipe” (UNO, Rotterdam, The Netherlands). Endotracheal intubation is performed, and rats are ventilated at 60 breaths per minute with a mixture of N2O/O2/isoflurane. Polyethylene tubes (PE-50; Becton Dickinson, St. Michielsgestel, The Netherlands) are flushed with heparin and placed via the right carotid artery in the aorta and in the right internal jugular vein. A 5-cm midline laparotomy is performed, and a supra pubic catheter is inserted to monitor urine production.

After an acclimatization period of 15 min, the rats are randomized into five different groups (eight rats per group): 1) sham, 2) HS, 3) HS with SEQ ID NO:6 (LQGVAPG) treatment, 4) HS with SEQ ID NO:7 (AQGVLPG) treatment, and 5) HS with SEQ ID NO:8 (LQGVLPG) treatment (HS/LAGV). Hemorrhagic shock (HS) is induced by blood withdrawal, reducing the circulating blood volume until a MAP of 40 mmHg is reached. This level of hypotension is maintained for 60 min. Rats receive a single intravenous bolus injection of 5 mg/kg body weight of either heptapeptide, or 0.9% NaCl solution 30 after the induction of HS. Sixty minutes after induction of HS, rats are resuscitated by four times their shed blood volume over a period of 30 min to normalize the MAP and monitored for another 120 min, after which they are killed. The rats receive no heparin before or during the experiment. Sham animals undergo the same surgical procedure as the HS animals but without blood withdrawal and administration of oligopeptide. Liver, lungs, ileum, and sigmoid are surgically removed at 180 min after HS induction, snap-frozen, and stored at -80° C. until assayed.

Leukocyte extravasation is characterized by increased expression of adhesion molecules such as E-selectin and intracellular adhesion molecule 1 (ICAM-1) on endothelial cells and hepatocytes. Up-regulation of these adhesion molecules facilitates tissue infiltration by leucocytes such as neutrophils, resulting in cell-mediated organ injury. Using a rat model of trauma and resuscitation, Q-ER heptapeptide given after the induction of trauma attenuates E-selectin mRNA transcript levels in the liver. In addition, Q-ER heptapeptide treatment prevents extravasation with neutrophil accumulation in the liver.

Example 19 Q-ER peptides provided with an elastin-receptor binding motif and enriched with amino acids that inhibit autophagy reduce renal dysfunction.

Short synthetic Q-ER peptides having been provided with an elastin receptor binding motif, (examples of which are shown in tables above) comprising distinct and selected amino acids preferentially activate mTOR or preferentially inhibit autophagy effect renal function. Acute effects of 140 minutes i.v. infusion of heptapeptide with SEQ ID NO:8 (LQGVLPG; 5 nmol x min(-1) x kg(-1) body wt) on renal function and urinary protein leakage are studied in anesthetized diabetic male rats two weeks after streptozotocin injection without insulin treatment. Streptozotocin-induced diabetic rats are studied with (N = 12) and without heptapeptide (N = 11) treatment. The two groups show a similarly elevated blood glucose concentration during the study. Age-matched normal rats serve as controls (N = 5). Glomerular filtration rate (GFR) is measured by inulin clearance in the basal state and during a 60-minute glycine infusion (0.22 mmol x min(-1) x kg(-1) body wt)-resembling a protein load challenge-to test the renal functional reserve in all three groups.

In the basal state, the non-heptapeptide-treated diabetic rats display increased GFR and increased total protein leakage when compared with normal rats. Whereas normal rats respond to glycine infusion with an increase in GFR, no increase occurs in diabetic rats not treated with heptapeptide. In diabetic rats given heptapeptide, this reduces the initial glomerular hyperfiltration prior to glycine infusion. Heptapeptide also restores normal renal functional reserve and results in lower total protein leakage compared with that in rats not given heptapeptide. Thus, short-term infusion of heptapeptide has beneficial effects on protein leakage and hyperfiltration and improves the renal functional reserve in rats.

1-30. (canceled)

31. A method of treating a subject in need thereof, the method comprising:
targeting cells having an elastin receptor complex associated with their surface with a molecule that specifically recognizes the elastin receptor complex,
wherein the molecule is provided with a source of autophagy inhibiting amino acids selected from the group consisting of alanine (in one letter code: A), glutamine (in one letter code: Q), glycine (in one letter code: G), valine (in one letter code: V), leucine (in one letter code: L), isoleucine (in one letter code: I), proline (in one letter code: P), and arginine (in one letter code: R).

32. The method according to claim 31, wherein the subject is in need of reduced autophagy.

33. The method according to claim 31, wherein the subject is in need of modified vascular permeability.

34. The method according to claim 31, wherein the subject is in need of enhanced tissue repair.

35. The method according to claim 31, wherein the subject is in need of a modulated immune response.

36. The method according to claim 31, wherein the molecule has an elastin peptide motif represented by xGxxPG, wherein x represents a naturally occurring amino acid.

37. The method according to claim 31, wherein the source of autophagy inhibiting amino acids is a peptide comprising the autophagy inhibiting amino acids.

38. The method according to claim 37, wherein the peptide comprising the autophagy inhibiting amino acids is selected from the group consisting of AQ, LQ, PQ, VQ, GQ, AQL, LQL, PQL, VQL, GQL, PLQ, LQG, PQV, VGQ, LQP, LQV, AQG, QPL, PQV, VGQ, GQG, SEQ ID NO:1 (LQGV), and SEQ ID NO:2 (AQGV).

39. The method according to claim 36, wherein xGxxPG is connected to the peptide comprising the autophagy inhibiting amino acids by a peptide bond.

40. The method according to claim 31, wherein the molecule is an antibody-like molecule selected from the group consisting of IgG, IgM, single chain antibodies, and FAB-or FAB′2-fragments.

41. The method according to claim 40, wherein the source of autophagy inhibiting amino acids is a peptide selected from the group consisting of AQ, LQ, PQ, VQ, GQ, AQL, LQL, PQL, VQL, GQL, PLQ, LQG, PQV, VGQ, LQP, LQV, AQG, QPL, PQV, VGQ, GQG, SEQ ID NO:1 (LQGV), and SEQ ID NO:2 (AQGV), connected to the antibody-like molecule through a peptide bond.

42. The method according to claim 40, wherein the antibody-like molecule is conjugated to the source of autophagy inhibiting amino acids.

43. The method according to claim 40, wherein the source of autophagy inhibiting amino acids is a lipid vesicle or liposome.

44. The method according to claim 43, wherein the lipid vesicle is a liposome comprising a oligopeptide selected from the group consisting of AQ, LQ, PQ, VQ, GQ, AQL, LQL, PQL, VQL, GQL, PLQ, LQG, PQV, VGQ, LQP, LQV, AQG, QPL, PQV, VGQ, GQG, SEQ ID NO: 1 (LQGV), and SEQ ID NO:2 (AQGV).

45. The method according to claim 43, wherein the molecule comprises amino acids selected from the group consisting of alanine (A), glutamine (Q), glycine (G), valine (V), leucine (L), isoleucine (I), proline (P), and arginine.

46. The method according to claim 33, wherein the molecule comprises amino acids selected from the group consisting of alanine (A), glutamine (Q), glycine (G), valine (V), leucine (L), isoleucine (I), proline (P), and arginine (R).

47. The method according to claim 34, wherein the molecule comprises amino acids selected from the group consisting of alanine (A), glutamine (Q), glycine (G), valine (V), leucine (L), isoleucine (I), proline (P), and arginine (R).

48. The method according to claim 35, wherein the molecule comprises amino acids selected from the group consisting of alanine (A), glutamine (Q), glycine (G), valine (V), leucine (L), isoleucine (I), proline (P), and arginine (R).

49. The method according to claim 45, wherein the molecule comprises an elastin peptide motif represented by xGxxPG, wherein x represents a naturally occurring amino acid.

50. The method according to claim 45, wherein the molecule comprises an antibody-like molecule, selected from the group consisting of IgG, IgM, single chain antibodies, and FAB- or FAB′2-fragments.

51. The method according to claim 45, wherein the source of autophagy inhibiting amino acids is a peptide selected from the group consisting of AQ, LQ, PQ, VQ, GQ, AQL, LQL, PQL, VQL, GQL, PLQ, LQG, PQV, VGQ, LQP, LQV, AQG, QPL, PQV, VGQ, GQG, SEQ ID NO: 1 (LQGV) and SEQ ID NO:2 (AQGV).

52. The method according to claim 51, wherein the molecule is connected to the peptide through a peptide bond.

53. The method according to claim 43, wherein the molecule comprises:
xGxxPG, wherein x represents a naturally occurring amino acid, and
a peptide selected from the group consisting of AQ, LQ, PQ, VQ, GQ, AQL, LQL, PQL, VQL, GQL, PLQ, LQG, PQV, VGQ, LQP, LQV, AQG, QPL, PQV, VGQ, GQG, SEQ ID NO:1 (LQGV), and SEQ ID NO:2 (AQGV).

54. A peptide having from 7 amino acids to at most 30 amino acids, the peptide comprising a sequence of the formula
ɸn xGxxPG, xGxxPG ɸn, or ɸn xGxxPG ɸm, wherein
x is a naturally occurring amino acid,
ɸ is an autophagy inhibiting amino acid,
n = an integer from 1 to 24, and
m is an integer from 1-23,
wherein n+m is no greater than 24.

55. The peptide of claim 54, wherein ɸn and/or ɸm comprise(s) AQ, LQ, PQ, VQ, GQ, AQL, LQL, PQL, VQL, GQL, PLQ, LQG, PQV, VGQ, LQP, LQV, AQG, QPL, PQV, VGQ, GQG, SEQ ID NO:1 (LQGV), or SEQ ID NO:2 (AQGV).

56. A method of reducing autophagy in a subject, the method comprising:
administering to the subject a pharmaceutical formulation comprising:
a peptide comprising xGxxPG, wherein x represents a naturally occurring amino acid,
a peptide selected from the group consisting of AQ, LQ, PQ, VQ, GQ, AQL, LQL, PQL, VQL, GQL, PLQ, LQG, PQV, VGQ, LQP, LQV, AQG, QPL, PQV, VGQ, GQG, SEQ ID NO: 1 (LQGV) and SEQ ID NO:2 (AQGV), and
at least one pharmaceutically acceptable excipient, so as to reduce autophagy in the subject.

57. A pharmaceutical formulation comprising:
the peptide of claim 54, and
at least one pharmaceutically acceptable excipient.

58. The pharmaceutical formulation of claim 57, further comprising insulin.

59. A method of treating impairment of pancreatic beta-cell function in a subject, the method comprising:
administering to the subject the pharmaceutical formulation of claim 58.

60. A method for producing the peptide of claim 54, the method comprising: synthesizing the peptide with an automated peptide synthesizer.


SEQUENCE LISTING (57 sequences)

SEQ ID NO: 1 - protein, 4 aa, Artificial Sequence
  Immunomodulatory peptide
LQGV

SEQ ID NO: 2 - protein, 4 aa, Artificial Sequence
  Immunomodulatory peptide
AQGV

SEQ ID NO: 3 - protein, 6 aa, Artificial Sequence
  Immunomodulatory peptide
QGVLPG

SEQ ID NO: 4 - protein, 6 aa, Artificial Sequence
  Immunomodulatory peptide
QGVAPG

SEQ ID NO: 5 - protein, 7 aa, Artificial Sequence
  Immunomodulatory peptide
AQGVAPG

SEQ ID NO: 6 - protein, 7 aa, Artificial Sequence
  Immunomodulatory peptide
LQGVAPG

SEQ ID NO: 7 - protein, 7 aa, Artificial Sequence
  Immunomodulatory peptide
AQGVLPG

SEQ ID NO: 8 - protein, 7 aa, Artificial Sequence
  Immunomodulatory peptide
LQGVLPG

SEQ ID NO: 9 - protein, 8 aa, Artificial Sequence
  Immunomodulatory peptide
AQGVAPGQ

SEQ ID NO: 10 - protein, 8 aa, Artificial Sequence
  Immunomodulatory peptide
LQGVAPGQ

SEQ ID NO: 11 - protein, 8 aa, Artificial Sequence
  Immunomodulatory peptide
AQGVLPGQ

SEQ ID NO: 12 - protein, 8 aa, Artificial Sequence
  Immunomodulatory peptide
LQGVLPGQ

SEQ ID NO: 13 - protein, 7 aa, Artificial Sequence
  Immunomodulatory peptide
LQGQAPG

SEQ ID NO: 14 - protein, 6 aa, Artificial Sequence
  Immunomodulatory peptide
QGQAPG

SEQ ID NO: 15 - protein, 8 aa, Artificial Sequence
  Immunomodulatory peptide
AQGQAPGQ

SEQ ID NO: 16 - protein, 8 aa, Artificial Sequence
  Immunomodulatory peptide
LQGQAPGQ

SEQ ID NO: 17 - protein, 8 aa, Artificial Sequence
  Immunomodulatory peptide
AQGQLPGQ

SEQ ID NO: 18 - protein, 8 aa, Artificial Sequence
  Immunomodulatory peptide
LQGQLPGQ

SEQ ID NO: 19 - protein, 6 aa, Artificial Sequence
  Immunomodulatory peptide
VGVAPG

SEQ ID NO: 20 - protein, 8 aa, Artificial Sequence
  Immunomodulatory peptide
LQVGLLPG

SEQ ID NO: 21 - protein, 8 aa, Artificial Sequence
  Immunomodulatory peptide
AQPGQLPG

SEQ ID NO: 22 - protein, 7 aa, Artificial Sequence
  Immunomodulatory peptide
AQGVQPG

SEQ ID NO: 23 - protein, 14 aa, Artificial Sequence
  Immunomodulatory peptide
VVGSPSAQDEASPL

SEQ ID NO: 24 - protein, 8 aa, Artificial Sequence
  Immunomodulatory peptide
LQGVQPGQ

SEQ ID NO: 25 - protein, 8 aa, Artificial Sequence
  Immunomodulatory peptide
AQGVQPGQ

SEQ ID NO: 26 - protein, 7 aa, Artificial Sequence
  Immunomodulatory peptide
VQGVLPG

SEQ ID NO: 27 - protein, 7 aa, Artificial Sequence
  Immunomodulatory peptide
VQGVAPG

SEQ ID NO: 28 - protein, 7 aa, Artificial Sequence
  Immunomodulatory peptide
GQGVLPG

SEQ ID NO: 29 - protein, 7 aa, Artificial Sequence
  Immunomodulatory peptide
GQGVAPG

SEQ ID NO: 30 - protein, 7 aa, Artificial Sequence
  Immunomodulatory peptide
LQGQLPG

SEQ ID NO: 31 - protein, 7 aa, Artificial Sequence
  Immunomodulatory peptide
VQGQLPG

SEQ ID NO: 32 - protein, 8 aa, Artificial Sequence
  Immunomodulatory peptide
VQGVLPGQ

SEQ ID NO: 33 - protein, 8 aa, Artificial Sequence
  Immunomodulatory peptide
VQGVAPGQ

SEQ ID NO: 34 - protein, 7 aa, Artificial Sequence
  Immunomodulatory peptide
AQGQLPG

SEQ ID NO: 35 - protein, 7 aa, Artificial Sequence
  Immunomodulatory peptide
GQGQLPG

SEQ ID NO: 36 - protein, 8 aa, Artificial Sequence
  Immunomodulatory peptide
GQGVLPGQ

SEQ ID NO: 37 - protein, 8 aa, Artificial Sequence
  Immunomodulatory peptide
GQGVAPGQ

SEQ ID NO: 38 - protein, 8 aa, Artificial Sequence
  Immunomodulatory peptide
VQGQLPGQ

SEQ ID NO: 39 - protein, 8 aa, Artificial Sequence
  Immunomodulatory peptide
GQGQLPGQ

SEQ ID NO: 40 - protein, 8 aa, Artificial Sequence
  Immunomodulatory peptide
LQGLQPGA

SEQ ID NO: 41 - protein, 8 aa, Artificial Sequence
  Immunomodulatory peptide
LQGAQPGQ

SEQ ID NO: 42 - protein, 8 aa, Artificial Sequence
  Immunomodulatory peptide
LQGLQPGQ

SEQ ID NO: 43 - protein, 8 aa, Artificial Sequence
  Immunomodulatory peptide
VQGLQPGQ

SEQ ID NO: 44 - protein, 8 aa, Artificial Sequence
  Immunomodulatory peptide
VQGVQPGQ

SEQ ID NO: 45 - protein, 8 aa, Artificial Sequence
  Immunomodulatory peptide
VQGAQPGQ

SEQ ID NO: 46 - protein, 8 aa, Artificial Sequence
  Immunomodulatory peptide
VQGGQPGQ

SEQ ID NO: 47 - protein, 8 aa, Artificial Sequence
  Immunomodulatory peptide
AQGLQPGQ

SEQ ID NO: 48 - protein, 8 aa, Artificial Sequence
  Immunomodulatory peptide
AQGAQPGQ

SEQ ID NO: 49 - protein, 8 aa, Artificial Sequence
  Immunomodulatory peptide
AQGGQPGQ

SEQ ID NO: 50 - protein, 8 aa, Artificial Sequence
  Immunomodulatory peptide
GQGLQPGQ

SEQ ID NO: 51 - protein, 8 aa, Artificial Sequence
  Immunomodulatory peptide
GQGVQPGQ

SEQ ID NO: 52 - protein, 8 aa, Artificial Sequence
  Immunomodulatory peptide
GQGAQPGQ

SEQ ID NO: 53 - protein, 8 aa, Artificial Sequence
  Immunomodulatory peptide
GQGGQPGQ

SEQ ID NO: 54 - DNA, 21 bp, Artificial Sequence
  Primer
ggtggtcatgccggggttcct

SEQ ID NO: 55 - DNA, 20 bp, Artificial Sequence
  Primer
atgttgctgcctgcactgtt

SEQ ID NO: 56 - DNA, 20 bp, Artificial Sequence
  Primer
ccatccagacattacctggc

SEQ ID NO: 57 - DNA, 21 bp, Artificial Sequence
  Primer
ccctcacacattccaggtggt